SEQUENCE LISTING (11 sequences)

SEQ ID NO: 1 - DNA, 19 bp, Artificial Sequence
  Description of Artificial Sequence Synthetic primer
aaactcttcgcggtctttc

SEQ ID NO: 2 - DNA, 20 bp, Artificial Sequence
  Description of Artificial Sequence Synthetic primer
cttaacgctttcgcctgttc

SEQ ID NO: 3 - DNA, 21 bp, Artificial Sequence
  Description of Artificial Sequence Synthetic primer
cacttacaacttcgccgtcct

SEQ ID NO: 4 - DNA, 23 bp, Artificial Sequence
  Description of Artificial Sequence Synthetic primer
gggagtttctatgggagctcaga

SEQ ID NO: 5 - DNA, 22 bp, Artificial Sequence
  Description of Artificial Sequence Synthetic probe
aactcatgggccgaggcaccaa

SEQ ID NO: 6 - DNA, 18 bp, Artificial Sequence
  Description of Artificial Sequence Synthetic primer
gctccctgccccagaagt

SEQ ID NO: 7 - DNA, 25 bp, Artificial Sequence
  Description of Artificial Sequence Synthetic primer
tgtcacaatgttcttgaagaaatgg

SEQ ID NO: 8 - DNA, 21 bp, Artificial Sequence
  Description of Artificial Sequence Synthetic probe
agcacggccggacccaagatg

SEQ ID NO: 9 - DNA, 26 bp, Artificial Sequence
  Description of Artificial Sequence Synthetic primer
ctcaactacatggtctacatgttcca

SEQ ID NO: 10 - DNA, 20 bp, Artificial Sequence
  Description of Artificial Sequence Synthetic primer
ccattctcggccttgactgt

SEQ ID NO: 11 - DNA, 26 bp, Artificial Sequence
  Description of Artificial Sequence Synthetic probe
tgactccactcacggcaaattcaacg

(19) United States
(12) Patent Application Publication
(10) Pub. No.: US 2012/0233713 A1
(43) Pub. Date: Sep. 13, 2012

(54) METHODS AND SYSTEMS FOR INDUCIBLE ABLATION OF NEURAL CELLS

(76) Inventors: Brian Popko, Monee, IL (US); Maria Traka, Chicago, IL (US)

(21) Appl. No.: 13/393,782

(22) PCT Filed: Sep. 2, 2010
(86) PCT No.: PCT/US10/47761

§ 371 Date: May 18, 2012

Related U.S. Application Data
(60) Provisional application No. 61/275,839, filed on Sep. 2, 2009.

(51) Int. Cl. G01N 33/53; C12N 5/07; C12Q 1/68; A01K 67/027

(52) U.S. Cl. 800/3; 800/13; 800/14; 800/18; 800/17; 435/325; 435/7.1; 435/6.11

(57) ABSTRACT

The present invention relates to methods and systems for inducible ablation of neural cells, in particular non-proliferating cells, such as oligodendrocytes and Schwann cells. The methods and systems include an animal model that can be specifically induced to display phenotypic traits or characteristics of a demyelination condition. The methods and systems disclosed herein are useful for drug screening, by identifying compounds or agents that promote remyelination or reversal of phenotypic traits or characteristics of demyelination conditions.

### RELATED APPLICATIONS

This application claims the benefit of U.S. Provisional Application No. 61/275,839, filed Sep. 2, 2009, which is incorporated by reference herein in its entirety.

### STATEMENT AS TO FEDERALLY SPONSORED RESEARCH

This invention was made with government support under NS027336 and NS034939 awarded by the National Institutes of Health. The government has certain rights in the invention.

### BACKGROUND

Neuronal demyelination is a deleterious condition characterized by a reduction of myelin in the nervous system. Myelin is a vital component of the central (CNS) and peripheral (PNS) nervous system, which encases the axons of neurons and forms an insulating layer known as the myelin sheath. The presence of the myelin sheath enhances the speed and integrity of nerve signals in the form of electric potentials propagating down the axon. The loss of myelin sheath produces significant impairment in sensory, motor and other types of functioning as nerve signals reach their targets either too slowly, asynchronously (for example, when some axons in a nerve conduct faster than others), intermittently (for example, when conduction is impaired only at high frequencies), or not at all.

Neural tissue comprises neurons and supporting or glial cells. Glial cells outnumber neurons by about ten to one in the mammalian brain. Glial cells can be divided into four types: astrocytes, oligodendrocytes, Schwann cells and microglial cells. The myelin sheath is formed by the plasma membrane or plasmalemma of a type of glial cells, namely oligodendrocytes in the CNS, and Schwann cells in the PNS. Myelinating oligodendrocytes have been identified at demyelinated lesions, indicating that demyelinated axons can be repaired with the newly synthesized myelin.

Neuronal demyelination is manifested in a large number of hereditary and acquired disorders of the CNS and PNS. These disorders include Multiple Sclerosis (MS), Progressive Multifocal Leukoencephalopathy (PML), Encephalomyelitis, Central Pontine Myelolysis (CPM), Anti-MAG Disease, Leukodystrophies, Adrenoleukodystrophy (ALD), Alexander's Disease, Canavan Disease, Krabbe Disease, Metachromatic Leukodystrophy (MLD), Pelizaeus-Merzbacher Disease, Refsum Disease, Cockayne Syndrome, Van der Knapp Syndrome, and Zellweger Syndrome, Guillain-Barre Syndrome (GBS), chronic inflammatory demyelinating polyneuropathy (CIDP), and multifocual motor neuropathy (MMN). For the vast majority of these disorders, there are no cures and few effective therapies.

Multiple sclerosis is a common demyelinating disease of the central nervous system. The disease is typically characterized clinically by relapses and remissions, leading eventually to chronic disability. The earlier phase of multiple sclerosis is usually characterized by the autoimmune inflammatory strike against myelin sheath leading to paralysis, lack of coordination, sensory disturbances and visual impairment. The subsequent chronic progressive phase of the disease is typically due to active degeneration of the myelin sheath and inadequate remyelination of the demyelinated lesions (Franklin, Nat. Rev. Neurosci. 3:705-714 (2002); Bruck et al., J. Neurol. Sci. 206:181-185 (2003); Compston et al., Lancet 359:1221-1231 (2002)).

Oligodendrocytes are believed to be the principal target cells of demyelinating disorders and that recovery from these disorders necessitates the restoration of the normal myelin by oligodendrocytes. However, remyelination is often an inefficient process leading to significant disability and/or death. Evidence suggests that the principal cellular mechanisms of remyelination can differ with developmental myelination (Franklin, Nat. Rev. Neurosci. 3:705-714 (2002); Balabanov et al., Nat. Neurosci. 8:262-264 (2005); Farhadi et al., J. Neurosci. 23:10214-10223 (2003); Ruffini et al., Am. J. Pathol. 165: 2167-2175 (2004); Arnett et al., Science 306:2111-2115 (2004); Stidworhty et al., Brain 127: 1928-1941 (2004)). Therefore understanding remyelination is one of the key aspects of identifying cause, effects and ameliorative options for myelin repair. Indeed, a major challenge in MS research is to understand the cause of remyelination failure and to devise ways of ameliorating its consequences. In recent years, several lines of evidence have suggested that the demyelinated lesions in MS are not deficient in oligodendrocyte progenitor cells (OPCs), rather that remyelination failure is associated with the insufficient repopulation of oligodendrocytes (Chang et al., J. Neurosci. 20: 6404-6412 (2000); Lucchinetti et al., Brain 122:2279-2295 (1999); Maeda et al., Ann. Neurol. 49:776-785 (2001)).

Currently, the ability to identify remyelinated axons is based on the premise that the myelin sheath of such axons tends to be thinner, such as through the use of electron microscopy (EM) analysis for identification. EM analysis can be prohibitively arduous and costly for the routine analysis of in vivo remyelination, especially in situations such as experimental autoimmune encephalomyelitis (EAE), where the demyelination and remyelination can not precisely localized to one particular locus.

Therefore, there remains a considerable need for methods and systems for inducible ablation of neural cells, such as myelinating cells, to facilitate the identification and elucidation of the molecular basis of neuronal remyelination. For example, there is a need for methods and systems for inducible ablation of neural cells using non-immune based methods. Methods and systems, such as through the use of animal models, can be used not only for elucidating the molecular basis of remyelination, but also can be used for developing biologically active agents effective for promoting remyelination. The animal models can also provide a much needed system for studying the phenotypic characteristics of demyelinating disorders and for identifying biologically active agents that reverse these phenotypes. The present invention satisfies these needs and provides related advantages as well.

### SUMMARY

The present invention provides methods and systems for time and tissue-specific inducible ablation of cells. In one aspect, the invention is to control the expression of a first heterologous sequence, wherein the expression is controlled by tissue specific promoter, and expression is blocked until unblocked by the addition of an inducing agent, allowing transcriptional access to the tissue specific promoter and expression of a first heterologous sequence encoding a protein which can then induce expression of a second heterologous sequence encoding a for protein which induces cell death. Such a coordinated system allows for control of time and location gene expression leading to cell death. In one aspect, cells are neural cells, such as myelinating cells. A non-human transgenic animal, said animal comprising: a) a first heterologous nucleotide sequence operably linked to a glial-cell specific promoter, wherein said first heterologous nucleotide sequence encodes a first heterologous protein and said first heterologous nucleotide sequence is stably expressed in said animal; and b) a second heterologous nucleotide sequence encoding a second heterologous protein, wherein said second heterologous protein is expressed upon activation of said first heterologous protein and induces death of a non-proliferating glial cell, wherein an initial activation of said first heterologous protein induces death of said non-proliferating glial cells in said animal and results in demyelination in said animal and yields one or more phenotypic changes characteristic of a demyelination condition; and wherein said one or more phenotypic changes is reversed after initial activation of said first heterologous protein. In some embodiments, the animal is a mammal, such as an animal selected from the group consisting of a mouse, rat, guinea pig, rabbit, dog, cat, pig, and monkey. For example, the animal can be a mouse that is at least approximately 5 weeks old, approximately 4 to 6 months old, or greater than 6 months old. The non-proliferating glial cells can be in the central nervous system (CNS), peripheral nervous system (PNS), or both. In some embodiments, the death of the non-proliferating glial cell results in demyelination in the CNS, PNS, or both.

In some embodiments, the non-human transgenic animal of the present invention comprises in its genome a first heterologous nucleotide sequence operably linked to a glial-cell specific promoter, wherein the first heterologous nucleotide sequence encodes a first heterologous protein, and the activity of the first heterologous protein is inducible. The activity, such as recombination, can be induced by an exogenous agent. The first heterologous protein can be a recombinase, such as a Cre recombinase or variant thereof. In some embodiments, the variant is a fusion protein, such as a fusion of Cre recombinase and a mutated ligand binding domain of an estrogen receptor. In such embodiments, the exogenous agent can be tamoxifen, or an analog thereof, such as when the fusion protein is CreER^{T} or CreER^{T2}. In some embodiments, the exogenous agent is administered to the animal more than once. In yet other embodiments, lipopolysaccharide (LPS) is administered to the animal prior to, concurrent with, or subsequent to the activation of the first heterologous protein or to the administration of the exogenous agent. The exogenous agent, the LPS, or both, can be administered focally or intraperitoneally, such as to the CNS or PNS. For example, the focal administration of the exogenous agent, LPS, or both can be to the brain, optic nerve, or spinal cord.

In some embodiments, the non-human transgenic animal of the present invention comprises in its genome a first heterologous nucleotide sequence operably linked to a glial-cell specific promoter. For example, the promoter can be a promoter of a gene selected from the group including but not limited to proteolipid protein (PLP), myelin basic protein (MBP), oligodendrocyte specific protein (OSP), myelin oligodendrocyte glycoprotein (MOG), ceramide galactosyltransferase (CGT), myelin associated glycoprotein (MAG), oligodendrocyte-myelin glycoprotein (OMG), cyclic nucleotide phosphodiesterase (CNP), NOGO, myelin protein zero (MPZ), peripheral myelin protein 22 (PMP22), or protein 2 (P2). Where desired, the particular embodiments, the promoter is a promoter of a gene selected from the group consisting of PLP, MBP, and CNP.

In another aspect, the non-human transgenic animal of the present invention comprises a second heterologous nucleotide sequence encoding a second heterologous protein, wherein the second heterologous protein induces death of non-proliferating glial cells upon activation of the first heterologous protein. In some embodiments, the second heterologous protein induces cell death. The second heterologous protein can be an exotoxin, such as a diptheria toxin or subunit thereof. For example, the second heterologous protein can be the A subunit of diptheria toxin (DT-A). The second heterologous protein can induce the death of both proliferating glials cells, such as oligodendrocyte projenitors or astrocytes, or of non-proliferating glial cells, such as mature oligodendrocytes or myelinating Schwann cells.

In some embodiments, the death of the non-proliferating glial cell in the non-human transgenic animal of the present invention results in demyelination and yields one or more phenotypic changes characteristic of a demyelination condition, such as, multiple sclerosis (MS). The one or more phenotypic changes can be wobbly gait, hind limb paralysis, tremors, weight loss, ataxia, or any combination thereof. In some embodiments, the one or more phenotypic changes characteristic of a demyelination condition is decreased motor control, balance, or CNS conduction. In some embodiments, the motor control or balance is measured by a rotarod behavioral assay or any other assay that provides spatial and temporal indices of posture and gait dynamics, such as by the DigiGait treadmill, and CNS conduction is measured by a spinal somatosensory evoked potential.

In yet another aspect of the present invention, the one or more phenotypic change characteristic of a demyelination condition is reversed after an initial activation of the first heterologous protein results in demyelination in the animal, such as in the CNS, PNS, or both. In some embodiments, the one or more phenotypic changes are reversed about 35 days or more after activation of the first heterologous protein. In yet other embodiments, the one or more phenotypic changes are reversed about 70 days or more after activation of the first heterologous protein.

Also provided herein is a cell of the non-human transgenic animal of the present invention. In some embodiments, the cell is an oligodendrocyte or Schwann cell. The cell can be a proliferating or non-proliferating cell.

The present invention also provides a method selecting a biologically active agent that promotes reversal of one or more phenotypic changes characteristic of a demyelination condition comprising: a) activating the first heterologous protein in the non-human transgenic animal of the present invention; b) administering a candidate agent to the animal; c) determining one or more phenotypic changes characteristic of a demyelination condition in the animal; and d) selecting the agent when the one or more phenotypic change is reversed more quickly as compared to a control animal not administered said candidate agent. The one or more phenotypic change characteristic of a demyelination conditions, such as MS, can be wobbly gait, hind limb paralysis, tremors, weight loss, ataxia, or any combination thereof. In some embodiments, the one or more phenotypic changes is decreased motor control, balance, or CNS conduction. In some embodiments, the motor control or balance is measured by a rotarod behavioral assay or any other assay that provides spatial and temporal indices of posture and gait dynamics, such as by the DigiGait treadmill, and CNS conduction is measured by a spinal somatosensory evoked potential.

Also provided herein is a method of selecting a biologically active agent that promotes remyelination comprising: a) activating the first heterologous protein in the non-human transgenic animal of the present invention; b) administering a candidate agent to the animal; c) determining remyelination in the animal; and d) selecting the agent when the animal displays increased remyelination as compared to a control non-transgenic animal not administered the candidate agent. In some embodiments, the remyelination is characterized by myelinated axons, by the expression of an oligodendrocyte cell marker, or a combination thereof. The oligodendrocyte cell marker can be selected from the group consisting of CC1, myelin basic protein (MBP), ceramide galactosyltransferase (CGT), myelin associated glycoprotein (MAG), myelin oligodendrocyte glycoprotein (MOG), oligodendrocyte-myelin glycoprotein (OMG), cyclic nucleotide phosphodiesterase (CNP), NOGO, myelin protein zero (MPZ), peripheral myelin protein 22 (PMP22), protein 2 (P2), galactocerebroside (GalC), sulfatide and proteolipid protein (PLP).

### BRIEF DESCRIPTION OF THE DRAWINGS

FIG. 1: depicts a schematic of a mouse comprising the ROSA26-eGFP-DTA allele that is crossed with a mouse with a PLP/CreER^{T} allele, generating a PLP/CreER^{T}; ROSA26-eGFP-DTA mouse. Cre-mediated excision of the floxed region is induced with tamoxifen in the PLP/CreER^{T}; ROSA26-eGFP-DTA mouse, resulting in DTA expression.

FIG. 2: illustrates PCR results confirming the expression of DTA message in the brain of tamoxifen treated PLP/CreER^{T}; ROSA26-eGFP-DTA mice using primers P1 and P2 as depicted in FIG. 1. Adult PLP/CreER^{T}; ROSA26-eGFP-DTA mice without induced expression of DTA (Control); and with induced DTA expression by tamoxifen (mut-D7, mut-D14, and mut-D21, representing the tamoxifen-treated mice 7 days, 14 days, and 21 days after the first tamoxifen injection (dpi), respectively) are depicted.

FIG. 3: illustrates cell death occurrence in PLP/CreER^{T}; ROSA26-eGFP-DTA tamoxifen-treated mice. (A) Increased numbers of TUNEL positive cell nuclei (arrows) were found in the corpus callosum area of the PLP/CreER^{T}; ROSA26-eGFP-DTA tamoxifen-treated mice at 5 dpi as compared to controls. (B) The same area showed increased numbers of cells stained positive for the active form of Caspase-3 (arrows), indicating that oligodendrocyte death occurs soon after Cre recombination is induces in these cells. Cell nuclei were counterstained with DAPI.

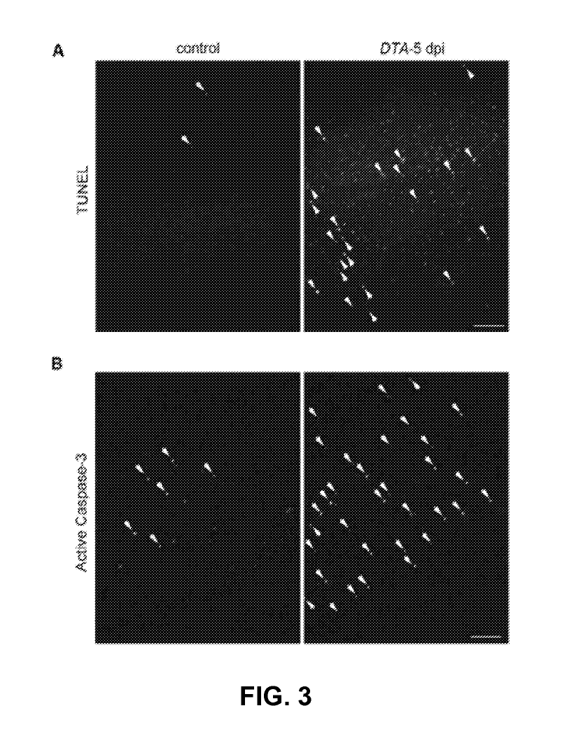

FIG. 4: illustrates low oligodendrocyte numbers in the CNS of the tamoxifen-treated PLP/CreER^{T}; ROSA26-eGFP-DTA mice 21 dpi. CC-1 staining in the cerebellum (upper panels) and spinal cord (lower panels) is decreased in the mouse 21 dpi (mut-D21) as compared to a control mouse.

FIG. 5: depicts a graph illustrating oligodendrocyte cell loss is a maximum in most CNS areas of tamoxifen-treated mice PLP/CreER^{T}; ROSA26-eGFP-DTA mice by 21 dpi (mut-D7, mut-D14, and mut-D21, representing the tamoxifen-treated mice 7, 14, and 21 dpi, respectively).

FIG. 6: depicts a graph illustrating a dramatic drop of (A) Plp and (B) Mbp mRNA levels that precedes oligodendrocyte cell loss in tamoxifen-treated mice PLP/CreER^{T}; ROSA26-eGFP-DTA mice at 7, 14, or 21 dpi, as compared to control mice.

FIG. 7: illustrates the impact of oligodendrocyte cell loss on CNS myelin is minimal in tamoxifen-treated PLP/CreER^{T}; ROSA26-eGFP-DTA mice 21 dpi as depicted by (A) toluidine blue staining (upper panels) and electron microscopy (EM, lower panels) of the spinal cord and (B) protein expression of MAG and MBP in the brain, as compared to control mice. Arrows in (A) point to white matter vacuoles that are generated by the splitting of the myelin sheath lamellae in the tamoxifen-treated PLP/CreER^{T}; ROSA26-eGFP-DTA mice 21 dpi.

FIG. 8: illustrates oligodendrocyte cell numbers recover to approximately normal in tamoxifen-treated PLP/CreER^{T}; ROSA26-eGFP-DTA mice by 70 dpi in the (A) brain stem, (B) cerebellum, (C) cervical cord gray matter, and (D) optic nerve. Quantification of CC-1 positive cells showed that oligodendrocyte numbers were significantly reduced in tamoxifen-treated PLP/CreER^{T}; ROSA26-eGFP-DTA mice at 21 dpi, then increased slightly in all areas at 35 dpi and reached values comparable to controls everywhere at 70 dpi. (n=4, *p<0.013, **p<0.006, two-tailed t-test). Graphs A-D indicate mean±standard deviation. (Grey=control, black=tamoxifen-treated PLP/CreER^{T}; ROSA26-eGFP-DTA mice).

FIG. 9: illustrates expression of (A) Plp and (B) Mbp mRNA levels in the brain of the tamoxifen-treated PLP/CreER^{T}; ROSA26-eGFP-DTA mice (“mutant”) show changes similar to oligodendrocyte cell numbers as illustrated in FIG. 8.

FIG. 10: illustrates the progression and repair of myelin defects in the CNS of tamoxifen-treated PLP/CreER^{T}; ROSA26-eGFP-DTA mice at 21, 35, and 70 days PI as compared to a control (mut-D21, mut-D35, and mut-D70, representing the tamoxifen-treated mice 21, 35, and 70 dpi, respectively).

FIG. 11: illustrates damage in the CNS myelin of the tamoxifen-treated PLP/CreER^{T}; ROSA26-eGFP-DTA mice is repaired by 70 dpi. PLP/CreER^{T}; ROSA26-eGFP-DTA mice (abbreviated as DTA herein) without induced expression of DTA (Control); and with induced DTA expression by tamoxifen (DTA-21 dpi, DTA-35 dpi, and DTA-70 dpi, representing the tamoxifen-treated mice 21 days, 35 days, and 70 days after the first tamoxifen injection, respectively) are depicted.

FIG. 12: illustrates increased microglia activation in the CNS of tamoxifen-treated PLP/CreER^{T}; ROSA26-eGFP-DTA mice. (A) CD11b marker in the cerebellum (CB) of tamoxifen-treated PLP/CreER^{T}; ROSA26-eGFP-DTA mice 35 dpi correlates with demyelination. (B-E) Total CNS cells were isolated from the brains and spinal cords of individual littermate control and tamoxifen-treated PLP/CreER^{T}; ROSA26-eGFP-DTA mice (n=8) at 35 dpi. The cells were enumerated and analyzed via flow cytometric analysis, gating on live cells for the present of CD45⁺ cells and gating on the CD45^{hi} versus CD45^{lo} populations for the presence of CD11b⁺cells. Flow plots from a representative littermate control mouse (B), and tamoxifen-treated PLP/CreER^{T}; ROSA26-eGFP-DTA mouse (DTA) (C) are presented. The total number of CD45^{hi} and CD45^{lo} cells (D), and the total number of CD45^{lo}/CD11b⁺ cells (E) into control versus DTA animals are shown. (Data presented as the average of the cell counts from 8 mice per group±S.E.M. **p<0.001 (D), **p<0.004 (E). Scale bar: 100 μm (A).

FIG. 13: illustrates numbers of adult oligodendrocyte precursor cells (OPCs) are increased in tamoxifen-treated PLP/CreER^{T}; ROSA26-eGFP-DTA mice 35 dpi as shown by PDGFRα positive cells in the brain stem and cerebellum.

FIG. 14: depicts a quantitative assessment of the defects and subsequent recovery of motor function in tamoxifen-treated PLP/CreER^{T}; ROSA26-eGFP-DTA mice (“mutant”) using the rotarod behavioral assay, which provides an estimate of the motor co-ordination and balance of the mice.

FIG. 15: depicts a schematic of spinal somatosensory evoked potentials (SEP) which is used to measure CNS conduction properties. SEP are recorded from the low lumbar (L4-L5) and mid-thoracic (T5-T6) vertebral levels by stimulating the tibial nerve at the ankle. The difference between the T5-%6 and L4-L5 SEP peak latencies (ΔLat) is used as an estimate of the CNS conductivity.

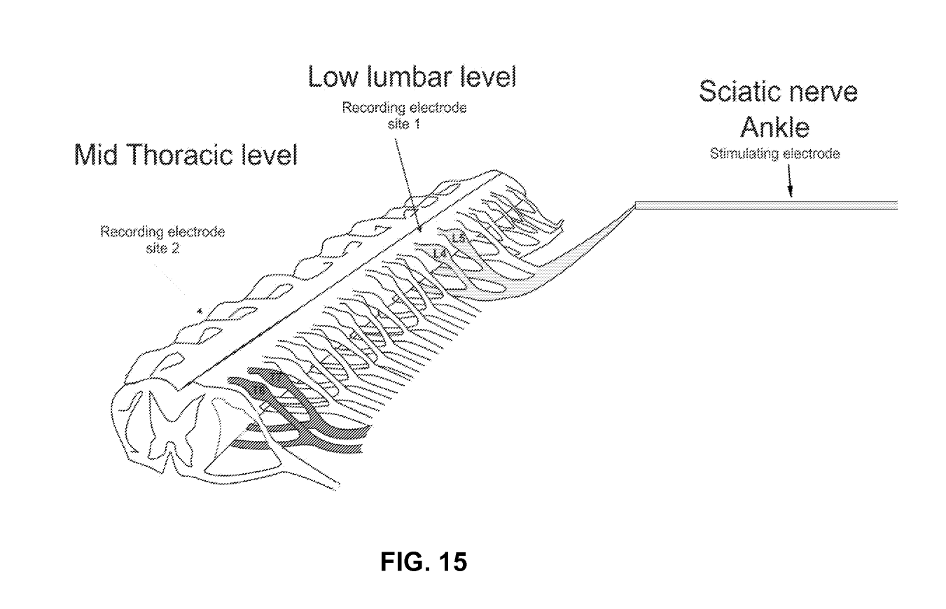

FIG. 16: illustrates SEP waveforms recorded from control and DTA mice at 35 and 70 dpi. Each SEP trace represents the averaging of 25-30 evoked responses recorded from the low lumbar level or mid thoracic level following stimulation of the tibial nerve at the ankle. No SEP evaluation was possible in DTA mice at 21 dpi which is likely due to the compromised myelin or neuronal function occurring both in the PNS and CNS.

FIG. 17: illustrates statistical analysis of the SEP parameters: peak latency (Lat) and Δlatency (ΔLat, difference between the T5-T6 and L4-L5 peak latencies), and amplitude (Amp). In DTA mice at 35 dpi, peak latencies are prolonged and Δlatencies are increased, while amplitudes are decreased, indicating severe conduction defects, both in the PNS and CNS of these mice. At 70-77 dpi, the amplitudes observed in DTA mice reach values comparable to controls, but though improved in comparison to 35 dpi values, a milder defect in peak latency time lingers. Graphs indicate mean±S.E.M. For 35 dpi, n=7-8, *p<0.005, **p<0.0001. For 70-77 dpi, n=6-7, *p<0.03; **p<0.008.

FIG. 18: illustrates quantification of myelin and axonal defects in the optic nerves of PLP/CreER^{T}; ROSA26-eGFP-DTA tamoxifen-treated mice. (A) EM analysis of the optic nerve revealed that the extensive myelin loss observable in PLP/CreER^{T}; ROSA26-eGFP-DTA tamoxifen-treated mice (“DTA”) at 56 dpi is repaired by 70 dpi, at which point many axons were found to be thinly myelinated. (B) Although the numbers of unmyelinated axons were significantly higher in PLP/CreER^{T}; ROSA26-eGFP-DTA tamoxifen-treated mice (“DTA”) at both time points, there were ˜70% fewer unmyelinated axons at 70 dpi as compared to 56 dpi (**p<1.30×10⁻⁵, n=3). (C) Linear regression analysis comparing the g-ratios and axonal diameters of myelinated axons in the optic nerve showed thinner myelin for all sizes of axons in PLP/CreER^{T}; ROSA26-eGFP-DTA tamoxifen-treated mice (“DTA”) at 70 dpi (black dots, n=3) as compared to controls (grey dots, n=3). (D) The total numbers of axons in the optic nerves of PLP/CreER^{T}; ROSA26-eGFP-DTA tamoxifen-treated mice (“DTA”) were found to be similar to control values at 21 and 70 dpi (p>0.27, n=3). Graphs in B and D indicate mean±S.D. Scale bar: 2 μm (A).

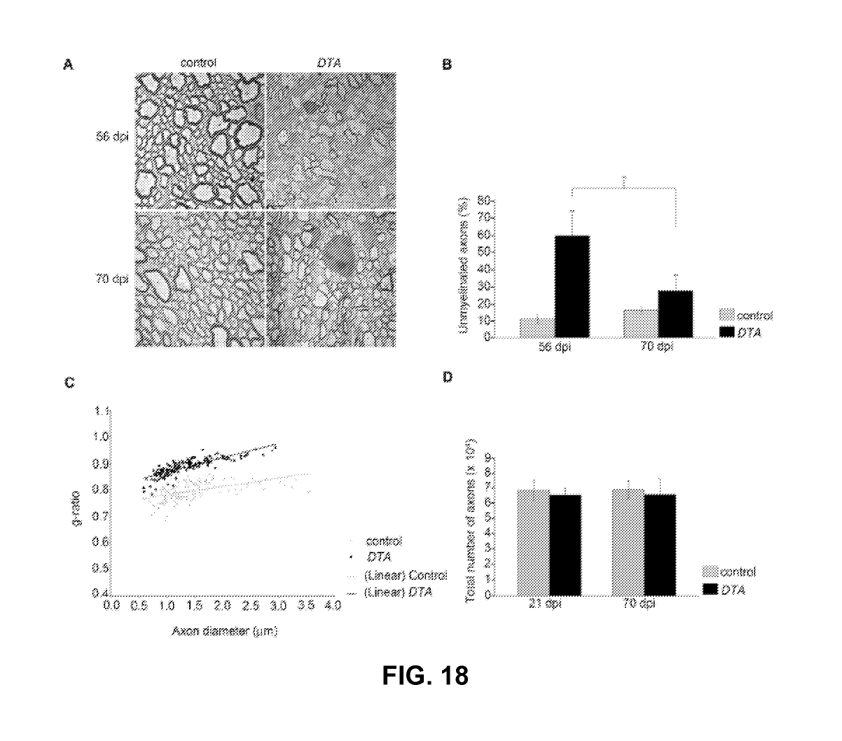

FIG. 19: illustrates a timeline of the progression of phenotypes characteristic of demyelination conditions such as multiple sclerosis and of remyelination.

### DETAILED DESCRIPTION OF THE EMBODIMENTS

Throughout this disclosure, various publications, patents and published patent specifications are referenced by an identifying citation. The disclosures of these publications, patents and published patent specifications are hereby incorporated by reference into the present disclosure to more fully describe the state of the art to which this invention pertains.

### General Techniques

The practice of the present invention employs, unless otherwise indicated, conventional techniques of immunology, biochemistry, chemistry, molecular biology, microbiology, cell biology, genomics and recombinant DNA, which are within the skill of the art. See Sambrook, Fritsch and Maniatis, MOLECULAR CLONING: A LABORATORY MANUAL, 3^{rd} edition (2001); CURRENT PROTOCOLS IN MOLECULAR BIOLOGY (F. M. Ausubel, et al. eds., (1987)); the series METHODS IN ENZYMOLOGY (Academic Press, Inc.): PCR 2: A PRACTICAL APPROACH (M. J. MacPherson, B. D. Hames and G. R. Taylor eds. (1995)), Harlow and Lane, eds. (1988) ANTIBODIES, A LABORATORY MANUAL, and ANIMAL CELL CULTURE (R. I. Freshney, ed. (1987)); B. K. C. Lo, ANTIBODY ENGINEERING: METHODS AND PROTOCOLS (2003); B. C. Chen, PCR CLONING PROTOCOLS (2010).

### DEFINITIONS

As used in the specification and claims, the singular form “a”, “an” and “the” include plural references unless the context clearly dictates otherwise. For example, the term “a cell” includes a plurality of cells, including mixtures thereof.

The terms “polynucleotide”, “nucleotide”, “nucleotide sequence”, “nucleic acid” and “oligonucleotide” are used interchangeably. They refer to a polymeric form of nucleotides of any length, either deoxyribonucleotides or ribonucleotides, or analogs thereof. Polynucleotides can have any three-dimensional structure, and can perform any function, known or unknown. The following are non-limiting examples of polynucleotides: coding or non-coding regions of a gene or gene fragment, loci (locus) defined from linkage analysis, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A polynucleotide can comprise modified nucleotides, such as methylated nucleotides and nucleotide analogs. If present, modifications to the nucleotide structure can be imparted before or after assembly of the polymer. The sequence of nucleotides can be interrupted by non-nucleotide components. A polynucleotide can be further modified after polymerization, such as by conjugation with a labeling component.

The terms “polypeptide”, “peptide” and “protein” are used interchangeably herein to refer to polymers of amino acids of any length. The polymer can be linear or branched, it can comprise modified amino acids, and it can be interrupted by non-amino acids. The terms also encompass an amino acid polymer that has been modified; for example, disulfide bond formation, glycosylation, lipidation, acetylation, phosphorylation, or any other manipulation, such as conjugation with a labeling component. As used herein the term “amino acid” refers to either natural and/or unnatural or synthetic amino acids, including glycine and both the D or L optical isomers, and amino acid analogs and peptidomimetics.

The term “heterologous” means derived from a genotypically distinct entity from the rest of the entity to which it is being compared. The term “heterologous” as applied to a polynucleotide or a polypeptide means that the polynucleotide or polypeptide is derived from a genotypically distinct entity from that of the rest of the entity to which it is being compared. For example, as applied to a nucleotide sequence or protein, the nucleotide sequence or protein is derived from a genotypically distinct entity from that of the rest of the entity to which it is being compared.

As used herein, “expression” refers to the process by which a polynucleotide is transcribed into mRNA and/or the process by which the transcribed mRNA (also referred to as “transcript”) is subsequently being translated into peptides, polypeptides, or proteins. The transcripts and the encoded polypeptides are collectedly referred to as “gene product.” If the polynucleotide is derived from genomic DNA, expression can include splicing of the mRNA in a eukaryotic cell.

The term “remyelinating” or “remyelination” refers to repair myelination or myelination that is not developmental.

A “subject,” “individual” or “patient” is used interchangeably herein, which refers to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

The “biologically active agents” that are employed in the animal model or cell culture assays described herein can be selected from the group consisting of a biological or chemical compound such as a simple or complex organic or inorganic molecule, peptide, peptide mimetic, protein (e.g. antibody), liposome, small interfering RNA, or a polynucleotide (e.g. anti-sense). Furthermore, such agents include complex organic or inorganic molecules can include a heterogeneous mixture of compounds, such as crude or purified plant extracts.

A “promoter element” is a regulatory sequence that promotes transcription of a gene that is linked to such a sequence. The regulatory sequence can include enhancer sequences or functional portions thereof.

A “control” is an alternative subject, cell or sample used in an experiment for comparison purpose.

A “floxed” DNA region refers to a region of DNA flanked by two lox sites, including variant lox sites, and the DNA region contains a transcription terminator (e.g., stop signal). The DNA region typically comprises a gene. For example, the “floxed” DNA can be a marker such as eGFP.

A “stoplight construct” refers to a gene construct comprising a first gene that is floxed that is further operably linked to a second gene. The “stoplight construct” can also be referred to as “stoplight cassette,” and can optionally be operably linked to a promoter sequence. Therefore, if the first floxed gene is removed through recombinase (e.g., Cre) mediated recombination, the second gene would be expressed. For example, the first floxed gene can be a fluorescent marker protein, such as eGFP, that is operably linked to a second gene encoding a toxin, such as DTA. The expression of the second gene, such as DTA, is inhibited because of the floxed region comprising the first region and a stop signal. When the floxed eGFP and stop signal is removed through recombination, the second gene, DTA, can be transcribed and expressed.

A “phenotype characteristic of a demyelination condition” refers to a phenotype characteristic of a demyelination condition that is ascertainable without analysis of axons, myelinating cells, or other molecular or cellular analyses, such as electron microscopy of axons, immunohistochemistry, or determining gene expression or protein expression of neural cells, such as oligodendrocytes or Schwann cells. For example, a phenotype change characteristic of a demyelination condition can be a decrease in motor control, balance, or CNS conduction, which can be measured by means such as a rotarod behavioral assay or spinal somatosensory evoked potential. Other phenotypes characteristic a demyelination condition include phenotypes such as wobbly gait, hind limb paralysis, tremors, weight loss, ataxia, or any combination thereof.

### Transgenic Animals

The present invention provides methods and systems for inducible ablation of neural cells. The model system is useful in elucidating mechanisms of remyelination, as well as development of therapeutic strategies for promoting remyelination. In one aspect, the system or animal model is a non-human transgenic animal capable of being induced to exhibit selective ablation of non-proliferating glial cells. In some embodiments, the specific ablation of non-proliferating glial cells results in demyelination in the animal.

Demyelination can be characterized by a decrease in myelinated axons in the nervous systems (e.g., the central or peripheral nervous system), or by a reduction in the levels of markers of myelinating cells. As used herein, myelinating cell refers to those cells capable of producing myelin which insulates axons in the nervous system. Exemplary myelinating cells are oligodendrocytes responsible for producing myelin in the central nervous system, and Schwann cells responsible for producing myelin in the peripheral nervous system. Morphologically, neuronal demyelination can be characterized by a loss of oligodendrocytes in the central nervous system or Schwann cells in the peripheral nervous system. It can also be determined by a decrease in myelinated axons in the nervous system, or by a reduction in the levels of oligodendrocyte or Schwann cell, CC1, markers. Exemplary marker proteins of oligodendrocytes or Schwann cells include, but are not limited to, myelin basic protein (MBP), ceramide galactosyltransferase (CGT), myelin associated glycoprotein (MAG), myelin oligodendrocyte glycoprotein (MOG), oligodendrocyte-myelin glycoprotein (OMG), cyclic nucleotide phosphodiesterase (CNP), NOGO, myelin protein zero (MPZ), peripheral myelin protein 22 (PMP22), protein 2 (P2), galactocerebroside (GalC), sulfatide and proteolipid protein (PLP). As such, the candidate agents identified by the subject method encompass substances that can inhibit the deleterious morphological characteristics of neuronal demyelination.

Demyelination can be also be characterized by the animal exhibiting one or more phenotypic changes characteristic of a demyelination condition. A phenotype change characteristic of a demyelination condition can be a decrease in motor control, balance, or CNS conduction, which can be measured by means such as a rotarod behavioral assay or spinal somatosensory evoked potential. Other phenotypes characteristic of a demyelination condition include phenotypes such as wobbly gait, hind limb paralysis, tremors, weight loss, ataxia, or any combination thereof.

The transgenic animal disclosed herein is capable of exhibiting one or more phenotypic changes characteristic of a demyelination condition, wherein the one or more phenotypic changes is reversed. For example, after the induction of selective ablation of the non-proliferating glial cells and the animal exhibits one or more phenotypic changes characteristic of a demyelination condition, the animal is capable of exhibiting a reversal of the one or more phenotypic changes characteristic of a demyelination condition. In some embodiments, the reversal can be a complete reversal, wherein the animal recovers from the one or more phenotypic changes characteristic of a demyelination condition such that the animal exhibits phenotypes that a wild-type animal or an animal with the same genetic background but not induced to exhibit selective ablation of non-proliferating glial cells exhibits. In other embodiments, a reversal of a phenotype is when the phenotype improves. For example, at timepoint A, an animal begins to show tremors; at timepoint B, the animal has increased tremors; and at timepoint C, the animal shows a phenotype similar to that as in timepoint A, which is an improved phenotype as compared to timepoint B. In another example, a reversal of a phenotype may be when there is a complete loss of a function, such as motor function or CNS conduction, and reversal is a gain in function, such as gain of motor function or CNS conduction. The gain can be a slight improvement in function. In some embodiments, the reversal can be regaining complete function, such as the to the same level of function as a wild-type animal or an animal with the same genetic background but not induced to exhibit selective ablation of non-proliferating glial cells.

The non-human transgenic animal capable of being induced to exhibit selective ablation of non-proliferating glial cells comprises a first heterologous nucleotide sequence that encodes a first heterologous protein and a second heterologous nucleotide sequence that encodes a second heterologous protein. The activity of the first heterologous protein, such as its expression and regulation of transcriptional events, can be inducible. This induction of activity of the first heterologous protein is also referred to herein as “activation” of such protein. In some embodiments, the first heterologous protein is a recombinase and its recombination activity is inducible. In some embodiments, the recombinase is a Cre recombinase, which recognizes the cognate recognition sequences, loxP sequences (i.e., loxP sites). Recognition sequences are known in the art, and represent particular DNA sequences which a protein, DNA, or RNA molecule (e.g., restriction endonuclease, a modification methylase, or a recombinase) recognizes and binds. For example, the recognition sequence for Cre recombinase is loxP which is a 34 base pair sequence comprised of two 13 base pair inverted repeats (serving as the recombinase binding sites) flanking an 8 base pair core sequence. (See Sauer, Curr. Opin. Biotech. 5:521-527 (1994)). Other examples of recognition sequences are the attB, attP, attL, and attR sequences which are recognized by the recombinase enzyme λ Integrase. attB is an approximately 25 base pair sequence containing two 9 base pair core-type Int binding sites and a 7 base pair overlap region. attP is an approximately 240 base pair sequence containing core-type Int binding sites and arm-type Int binding sites as well as sites for auxiliary proteins IHF, FIS, and Xis. (See Landy, Curr. Opin. Biotech. 3:699-707 (1993)). Such sites can also engineered according to the present invention to enhance recombination utilizing methods and products as known in the art such as disclosed in the disclosure by Hartley et al., U.S. Patent Application Publication No. 20060035269.

The Cre recombinase can be wild type or a variant of the wild type. In some embodiments, the activity of the Cre recombinase is inducible in the transgenic animal (or transgenic cells). Variant Cre recombinases have broadened specificity for the site of recombination. Specifically, the variants mediate recombination between sequences other than the loxP sequence and other lox site sequences on which wild type Cre recombinase is active. In general, the disclosed Cre variants mediate efficient recombination between lox sites that wild type Cre can act on (referred to as wild type lox sites), between variant lox sites not efficiently utilized by wild type Cre (referred to as variant lox sites), and between a wild type lox site and a variant lox site. For example, the Cre variants can be used in any method or technique where Cre recombinase (or other, similar recombinases such as FLP) can be used. In addition, the Cre variants allow different alternative recombinations to be performed since the Cre variants allow much more efficient recombination between wild type lox sites and variant lox sites. Control of such alternative recombination can be used to accomplish different sequential recombinations to achieve results not possible with wild type Cre recombinase. Variant Cre recombinases are known in the art, such as disclosed in the disclosure of U.S. Pat. No. 6,890,726, which is herein incorporated by reference in its entirety.

In some embodiments, the first heterologous sequence encodes a recombinase engineered to be active when induced by an exogenous agent. The inducibility of Cre activity can be controlled. In some embodiments, the Cre protein can be a fusion of the Cre recombinase with a mutated version of the ligand-binding domain of the progesterome receptor (e.g. Kellendonk et al., J. Mol. Biol. 285:175-182 (1999)) or the estrogen receptor (e.g. Feil et al., Proc. Natl. Acad. Sci. 93:10887-10890 (1996); Feil et al., Biochem. Biophsys. Res. Commun. 237:752-757 (1997)) and the activity induced by an exogenous agent. For example, the latter fusions result in the Cre fusion, CreER^{T} or CreER^{T2}, where the recombination activity is controlled by a ligand through the localization of the Cre protein. In the absence of ligand, CreER^{T} (or CreER^{T2}) is cytoplasmic. However, following administration of a synthetic steroid hormone (tamoxifen, or a variant or analog thereof, such as 4-hydroxy (OH)-tamoxifen), the CreER^{T} (or CreER^{T2}) protein translocates into the nucleus where it is functional (i.e., tamoxifen-inducible). In other embodiments, other inducible systems may be used, such as a Tet-On and Tet-Off system (Clontech Laboratories, Inc) expression system in which induction occurs through the addition of an antibiotic, such as tetracycline.

The first heterologous sequence can be stably expressed in the non-human transgenic animal. For example, the first heterologous sequence encoding a recombinase integrated into the genome of the transgenic animal, such as by being under the control to an endogenous promoter. The first heterologous sequence can be operably linked to a glial-cell specific promoter, such as an endogenous glial-cell specific promoter. The promoter can be specific for astroglia, oligodendrocytes or Schwann cells. In some embodiments, the promoter is for a gene that is highly expressed in mature, differentiating, or non-proliferating glial cells, such as mature oligodendrocytes or Schwann cells. The promoter can be for a gene that is highly expressed in myelinating cells. The promoter can be for proteolipid protein (PLP), myelin basic protein (MBP), oligodendrocyte specific protein (OSP), myelin oligodendrocyte glycoprotein (MOG), ceramide galactosyltransferase (CGT), myelin associated glycoprotein (MAG), oligodendrocyte-myelin glycoprotein (OMG), cyclic nucleotide phosphodiesterase (CNP), NOGO, myelin protein zero (MPZ), peripheral myelin protein 22 (PMP22), protein 2 (P2). In some embodiments, the first heterologous sequence can be operably linked to a promoter specific to proliferating oligodendrocyte progenitors, such as platelet derived growth factor alpha receptor (PDGFRα), a promoter specific to astrocytes, such as glial fibrillary acidic gene (GFAP), or a promoter specific to neurons, such as neuron specific enolase (NSE), tyrosine hydroxylase, and BSF1. Thus, expression and activity of the first heterologous protein, such as a recombinase, can be cell-specific.

In the non-human transgenic animal disclosed herein, the activity of the first heterologous protein can induce expression of the second heterologous protein, such as by inducing recombination to remove a stop signal and allow expression of the second heterologous protein, such as a toxin. The expression of the second heterologous protein, or toxin, can then induce cell death. As expression and activity of the first heterologous protein, such as a recombinase, can be cell-specific, the activity of the first heterologous protein can induce expression of the second heterologous protein in a cell-specific manner. For example, the second heterologous sequence can encode the second heterologous protein, which induces cell death, and its expression, and resulting cell death can be cell-type specific. For example, expression of the second heterologous protein can specifically in neural cells, such as glial cells. In some embodiments, expression of the second heterologous protein can induce the cell-specific death, or cell-specific ablation, of oligodendrocytes, Schwann cells, or astrocytes. In some embodiments, the glial cells are non-proliferating glial cells, such as in the CNS, PNS, or both, wherein death of the non-proliferating glial cell results in demyelination in the CNS, PNS, or both.

As described, the second heterologous sequence can encode a toxin or exotoxin. For example, the toxin can be a diptheria toxin or subunit thereof, such as the A subunit. In some embodiments, the expression of the second heterologous sequence is repressed because of a floxed upstream region that comprises a termination or stop signal for transcription. Therefore, when the floxed upstream region comprising the stop signal is removed, such as by recombination (e.g., by Cre recombinase, such as CreER^{T} or CreER^{T2} encoded by the first heterologous sequence and activated by an exogenous agent), the second heterologous sequence can be transcribed and the protein expressed.

In another embodiment, the same heterologous sequence encodes a toxin or exotoxin and contains a cell-specific promoter. For example, the toxin can be a diptheria toxin or subunit thereof, such as the A subunit. In some embodiments, the promoter contains an upstream enhancer region which is blocked and once released after the addition of an inducer, as in the pdual expression vector system (Strategene), expression of the heterologous sequence which encodes for a toxin or exotoxin occurs.

In some embodiments, the floxed region includes one or more markers, such as fluorescent protein markers or drug-resistant markers. Non-exclusive examples of marker genes that can be used in the present invention include reef coral fluorescent proteins (RCFPs), HcRed1, AmCyan1, AsRed2, mRFP1, DsRed1, jellyfish fluorescent protein (FP) variants, red fluorescent protein, green fluorescent protein (GFP), blue fluorescent protein, luciferase, GFP mutant H9, GFP H9-40, eGFP, tetramethylrhodamine, Lissamine, Texas Red, EBFP, ECFP, EYFP, Citrine, Kaede, Azami Green, Midori Cyan, Kusabira Orange and naphthofluorescein, or enhanced functional variants thereof. Many genes encoding fluorophore proteins markers are known in the art, which are capable of use herein (See for example website: <cgr.harvard.edu/thornlab/gfps.htm>). Mutated version of fluorescence proteins that emit light of greater intensity or which exhibit wavelength shifts can also be utilized in the compositions and methods of the present invention; such variants are known in the art and commercially available. (See for example Clontech Catalogue, 2005).

In yet other embodiments, the second heterologous sequence encoding the toxin is operably linked to a glial-cell specific promoter, such as an endogenous glial-cell specific promoter. The promoter can be specific for astroglia, oligodendrocytes or Schwann cells. In some embodiments, the promoter is for a gene that is highly expressed in mature, differentiating, or non-proliferating glial cells, such as mature oligodendrocytes or Schwann cells. The promoter can be for a gene that is highly expressed in myelinating cells. The promoter can be for the proteolipid protein (PLP), myelin basic protein (MBP), oligodendrocyte specific protein (OSP), myelin oligodendrocyte glycoprotein (MOG), ceramide galactosyltransferase (CGT), myelin associated glycoprotein (MAG), oligodendrocyte-myelin glycoprotein (OMG), cyclic nucleotide phosphodiesterase (CNP), NOGO, myelin protein zero (MPZ), peripheral myelin protein 22 (PMP22), or protein 2 (P2). In some embodiments, the second heterologous sequence is operably linked to a promoter specific to proliferating oligodendrocyte progenitors, such as platelet derived growth factor alpha receptor (PDGFRα), a promoter specific to astrocytes, such as glial fibrillary acidic gene (GFAP), or a promoter specific to neurons, such as neuron specific enolase (NSE), tyrosine hydroxylase, and BSF1.

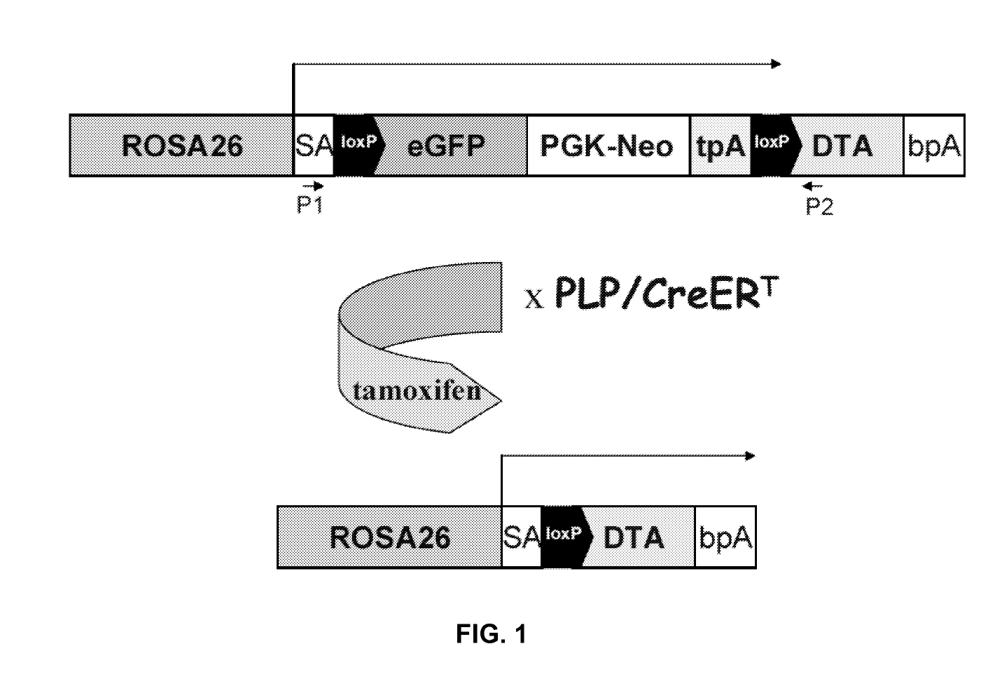

In some embodiments, the second heterologous sequence is a component of a stoplight cassette, such as shown in FIG. 1, where the floxed region includes eGFP and the second heterologous sequence encodes DTA. The stoplight cassette can be stably integrated into the genome of a transgenic mouse, such as into the ROSA26 locus (see e.g. FIG. 1). A transgenic animal comprising a first heterologous nucleotide sequence and a second heterologous nucleotide sequence in its genome can be generated, by mating a first animal comprising the first heterologous sequence with a second animal comprising the second heterologous sequence, such as depicted in FIG. 1. A transgenic animal comprising a stoplight cassette with floxed eGFP upstream of DTA integrated into the ROSA26 locus (ROSA26-eGFP-DTA) is mated with a transgenic animal comprising CreER^{T} under the control of the endogenous PLP promoter (PLP/CreER^{T}), which can result in an animal comprising the stoplight cassette and CreER^{T} under the control of the endogenous PLP promoter (PLP/CreER^{T}; ROSA26-eGFP-DTA). DTA is not expressed as the floxed region comprises a transcription termination signal (e.g. stop codon). However upon treatment with tamoxifen, CreER^{T} is activated and the floxed region is recombined such that the transcription termination signal is removed and DTA can be transcribed and expressed. Furthermore, since CreER^{T} activity is inducible, DTA expression can be effected in a time-controlled manner, as well as a cell-specific manner, resulting in specific ablation, or cell death, of specific cell types. For example, in the PLP/CreER^{T}; ROSA26-eGFP-DTA mice, there is specific ablation of PLP-expressing cells when the mice are treated with tamoxifen. Timing of the ablation can be controlled through the administration of the tamoxifen.

The transgenic animals, such as the ROSA26-eGFP-DTA animal and the PLP/CreER^{T} animal, can be designed utilizing gene targeting techniques known in the art (see e.g. Ivanova et al., Genesis, 43:129-135 (2005); Doerflinger et al., Genesis 35:63-72 (2003)). Gene targeting represents the directed modification of a chromosome locus by homologous recombination with an exogenous DNA sequence homologous with the targeted endogenous sequence. A distinction is made between different types of gene targeting. Thus, gene targeting can be used to modify, and usually increase, the expression of one or several endogenous genes, or to replace an endogenous gene by an exogenous gene, or to place an exogenous gene under the control of elements regulating the gene expression of the particular endogenous gene that remains active. In this case, gene targeting is called “Knock-in” (KI). Alternatively, gene targeting can be used to reduce or eliminate the expression of one or several genes, and this type of gene targeting is called “Knock-out” (KO) or “Knock-down” (KD) (See, e.g., Bolkey et al., Ann. Rev. Genet. 23:199-225 (1989)).

Methods of generating transgenic cells according to the invention are well known to those skilled in the art. Various techniques for transfecting mammal cells have been described (Gordon., Intl. Rev. Cytol. 115: 171-229 (1989)). For example, the transgenes disclosed herein and used for generating the transgenic animals can include in a linearized or non-linearized vector or in the form of a vector fragment, can be introduced into the host cell by standard methods for example such as micro-injection into the nucleus (U.S. Pat. No. 4,873,191), transfection by precipitation with calcium phosphate, lipofection, electroporation (Lo, Mol. Cell. Biol. 3:1803-1814 (1983)), thermal shock, transformation with cationic polymers (PEG, polybrene, DEAE-Dextran, etc.), viral infection (Van der Putten et al., Proc. Natl. Acad. Sci USA 82, 6148-6152 (1985)), sperm (Lavitrano et al., Cell 57:717-723 (1989)).

A transgenic animal can be engineered by insertion of a genetic construct into the pronucleus (such as the male pronucleus) of a mammalian zygote, allowing stable genomic integration to occur naturally. The zygote can then be transferred to a receptive uterus, and allowed to develop to term. While the mouse can be used, other species, such as rats, rabbits and other non-human animals are also potential candidates for pronuclear insertion. The genetic construct which renders the zygote transgenic comprises a gene construct that targets an endogenous gene to be exploited (e.g., PDGFα receptor gene), which gene can be mutated and/or further modified to comprise desired elements (e.g., a exogenous promoter/enhancer element and/or a gene of interest).

Heterologous DNA can also be introduced into fertilized mammalian ova as well. For instance, totipotent or pluripotent stem cells can be transformed by microinjection, calcium phosphate mediated precipitation, liposome fusion, retroviral infection or other means. The transformed cells are then introduced into the embryo, and the embryo will then develop into a transgenic animal. In one embodiment, developing embryos are infected with a viral vector containing a desired transgene so that the transgenic animals expressing the transgene can be produced from the infected embryo. In another embodiment, a desired transgene is coinjected into the pronucleus or cytoplasm of the embryo, preferably at the single cell stage, and the embryo is allowed to develop into a mature transgenic animal. These and other variant methods for generating transgenic animals are well established in the art and hence are not detailed herein. See, for example, U.S. Pat. Nos. 5,175,385 and 5,175,384.

In one or more aspects of the invention disclosed herein, a desired transgene can be integrated as a single copy or in concatamers, e.g., head-to-head tandems or head-to-tail tandems. The desired transgene can also be selectively introduced into and activated in a particular tissue or cell type, preferably cells within the central nervous system. The regulatory sequences required for such a cell-type specific activation will depend upon the particular cell type of interest, and will be apparent to those of skill in the art. Preferably, the targeted cell types are located in the nervous systems, including the central and peripheral nervous systems.

As noted above, transgenic animals can be broadly categorized into two types: “knockouts” and “knockins”. A “knockout” has an alteration in the target gene via the introduction of transgenic sequences that result in a decrease of function of the target gene, preferably such that target gene expression is insignificant or undetectable. A “knockin” is a transgenic animal having an alteration in a host cell genome that results in an augmented expression of a target gene, e.g., by introduction of an additional copy of the target gene, or by operatively inserting a regulatory sequence that provides for enhanced expression of an endogenous copy of the target gene. The knock-in or knock-out transgenic animals can be heterozygous or homozygous with respect to the target genes. Both knockouts and knockins can be “bigenic”. Bigenic animals have at least two host cell genes being altered. A some bigenic animal carries a transgene encoding a neural cell-specific recombinase and another transgenic sequence that encodes neural cell-specific marker genes. The transgenic animals of the present invention can broadly be classified as Knockins.

The transgenic animals disclosed herein include but are not limited to mammals, such as primates and rodents. Non-limiting examples include rats, mice, guinea pigs, cats, dogs, rabbits, pigs, goats, sheep, horses, cows, llamas, and monkeys. In one embodiment, the animal is a rodent. In yet another embodiment, the animal is a mouse, such as a young or old mouse. For example, the mice can be at least approximately 5, 6, 7, 8, 9, 10, 11, or 12 weeks old. The mice can be between approximately 3 to 8 weeks old, approximately 5 to 7 weeks old, or approximately 6 to 8 weeks old. In some embodiments, the mice are at least approximately 4 to 6 months old. In some embodiments, the mice are adults and greater than 6 months old. In some embodiments, the mice are aged or elderly mice. The mice can be at least approximately 7, 8, 9, 10, 11, 12, 15, 18, 21, or 24 months old. In some embodiments, the mice are between approximately 5 to 7 months old, 6 to 9 months old or 9 to 12 months old. In another embodiment, the animal is from a simian species. In yet another embodiment, the animal is a marmoset monkey, which can be utilized in examining neurological diseases (e.g., Eslamboi, Brain Res. Bull. 68:140-149 (2005); Kirik et al., Proc. Natl. Acad. Sci. 100:2884-2889 (2004)). The transgenic animals disclosed herein may also include but are not limited to non-mammals, such as fish, birds, and insects. Non-limiting examples include zebra fish, chickens and flies.

The transgenic animals disclosed here provide a system that can be utilized in assaying remyelination. Such a model system will provide insights into elucidating mechanisms of remyelination, as well as development of therapeutic strategies for promoting remyelination. As described above, the transgenic animals can be temporally controlled to ablate cells in a cell-specific manner by inducing activation of a first heterologous protein, which in turn induces expression of a second heterologous protein that induces cell death. The cell death can be cell type specific as the expression of the first heterologous protein can be under the control of cell-type specific promoter or regulatory sequence, in particular those available for expression in the central or peripheral nervous systems.

For example, the expression of the first heterologous protein can be under the control of neural cell-type specific promoter, such as disclosed in U.S. Patent Application Publication No. 2003/0110524; See also, the website <chinook.uoregon.edu/promoters.html>. The transcriptional regulatory sequence can include, but not be limited to, transcriptional regulatory sequences selected from the genes encoding the following proteins: the PDGFRα, proteolipid protein (PLP), the glial fibrillary acidic gene (GFAP), myelin basic protein (MBP), neuron specific enolase (NSE), oligodendrocyte specific protein (OSP), myelin oligodendrocyte glycoprotein (MOG) and microtubule-associated protein 1B (MAP1B), Thy1.2, ceramide galactosyltransferase (CGT), myelin associated glycoprotein (MAG), oligodendrocyte-myelin glycoprotein (OMG), cyclic nucleotide phosphodiesterase (CNP), NOGO, myelin protein zero (MPZ), peripheral myelin protein 22 (PMP22), protein 2 (P2), tyrosine hydroxylase, BSF1, dopamine 3-hydroxylase, Serotonin 2 receptor, choline acetyltransferase, galactocerebroside (GalC), and sulfatide.

In some embodiments, the regulatory sequence or promoter is specific for glial cells such as astroglia, oligodendrocytes or Schwann cells. In some embodiments, the promoter is for a gene that is highly expressed or specifically expressed in mature, differentiating, or non-proliferating glial cells, such as mature oligodendrocytes or Schwann cells. The promoter can be for a gene that is highly expressed or specifically expressed in myelinating cells. In some embodiments, the promoter or regulatory sequence is, but not limited to, proteolipid protein (PLP), myelin basic protein (MBP), oligodendrocyte specific protein (OSP), myelin oligodendrocyte glycoprotein (MOG), ceramide galactosyltransferase (CGT), myelin associated glycoprotein (MAG), oligodendrocyte-myelin glycoprotein (OMG), cyclic nucleotide phosphodiesterase (CNP), NOGO, myelin protein zero (MPZ), peripheral myelin protein 22 (PMP22), or protein 2 (P2). In some embodiments, the promoter is for PLP, MBP, and CNP. In some embodiments, the promoter is for a gene specific to proliferating oligodendrocyte progenitors, such as platelet derived growth factor alpha receptor (PDGFRα), a promoter specific to astrocytes, such as glial fibrillary acidic gene (GFAP), or a promoter specific to neurons, such as neuron specific enolase (NSE), tyrosine hydroxylase, and BSF1.

In some embodiments, the first heterologous sequence encodes a Cre recombinase, such as CreER^{T2} or CreER^{T2} and is under the control of an aforementioned promoter or regulatory sequence.

In some embodiments, the regulatory sequence can be altered or modified to enhance expression (i.e., increase promoter strength). For example, intronic sequences comprising enhancer function can be utilized to increase promoter function. The myelin proteolipid protein (PLP) gene comprises an intronic sequence that functions as an enhancer element. This regulatory element/region ASE (for antisilencer/enhancer) is situated approximately 1 kb downstream of exon 1 DNA and encompasses nearly 100 bp. See, Meng et al., J. Neurosci. Res. 82:346-356 (2005).

As such, the first heterologous protein, such as a Cre recombinase (e.g. CreER^{T2} or CreER^{T2}) can be expressed and activated in specific cell types and induce expression of the second heterologous protein, such as a toxin, in the same specific cell types. For example, the Cre recombinase (e.g. CreER^{T2} or CreER^{T2}) is expressed in oligodendrocytes by being under the control or regulation of an oligodendrocyte-specific promoter or regulatory sequence. When the Cre recombainse is activated by tamoxifen, expression of the second heterologous protein, such as a toxin such as DTA, is induced in oligodendrocytes. The toxin can then promote cell death of the oligodendrocytes. Alternatively, the Cre recombinase (e.g. CreER^{T2} or CreER^{T2}) can be expressed in Schwann cells by being under the control or regulation of a Schwann cell-specific promoter or regulatory sequence. The Cre recombinase can be activated by tamoxifen, which induces expression of the toxin in Schwann cells, causing cell death of the Schwann cells. The cell death of the myelinating cells, such as oligodendrocytes, Schwann cells, or both, can result in demyelination in the CNS, PNS, or both, of the animal.

The first heterologous protein can also be activated in a temporal manner, and thus, the resulting ablation or cell death can be temporally controlled. For example, the first heterologous protein can be activated in non-proliferating cells, such as mature or differentiated, neural cells. In some embodiments, the activation of the first heterologous protein is in mature, or non-proliferating, cells of the CNS, PNS, or both, resulting in expression of the second heterologous protein and subsequent cell death of the mature, or non-proliferating, cells. In some embodiments, the ablation is of non-proliferating myelinating cells, such as oligodendroctyes or Schwann cells. The activation can be temporally controlled by an exogenous agent, such as administration of the exogenous agent that activates the first heterologous protein. The exogenous agent can be administered to the animal when the glial cells are in the proliferating stage or non-proliferating stage. For example, the exogenous agent can be administered to the animal when the Schwann cells or oligodendrocytes are in the proliferating stage. In some embodiments, the exogenous agent is administered to the animal when the Schwann cells or oligodendrocytes are mature or non-proliferating. For example, in a mouse, the exogenous agent is administered when the mouse is at least approximately 5, 6, 7, 8, 9, 10, 11, or 12 weeks old. In some embodiments, the mice are treated with the exogenous agent when the mice are between approximately 3 to 8 weeks old, approximately 5 to 7 weeks old, or approximately 6 to 8 weeks old. In some embodiments, the mice are at least approximately 4 to 6 months old. In yet other embodiments, the mice can be at least approximately 7, 8, 9, 10, 11, 12, 15, 18, 21, or 24 months old. In some embodiments, the mice are between approximately 5 to 7 months old, 6 to 9 months old or 9 to 12 months old.

In one aspect, the invention is to control the expression of a first heterologous sequence, wherein the expression is controlled by tissue specific promoter, and expression is blocked until unblocked by the addition of an inducing agent, allowing transcriptional access to the tissue specific promoter and expression of a first heterologous sequence encoding a protein which can then induce expression of a second heterologous sequence encoding a for protein which induces cell death. Such a coordinated system allows for control of time and location gene expression leading to cell death.

In some embodiments, the transgenic animal comprises a first heterologous sequence encoding an inducible Cre recombinase such as CreER^{T}, which is under the control of an endogenous cell-specific promoter. The animal further comprises a second heterologous sequence encoding a toxin such as DTA, which is downstream of a floxed region comprises a transcription termination signal (e.g. stop codon). The animal expresses CreER^{T} in oligodendrocytes, however, without being bound by theory, the CreER^{T} is unable to translocate into the nucleus and perform recombination. As a result, the animal does not express DTA. However, upon treatment with tamoxifen, CreER^{T} is activated and the floxed region comprising the transcription termination signal is removed and DTA can be transcribed and expressed, which induces cell death.

In some embodiments, the transgenic animal comprises a first heterologous sequence encoding an inducible Cre recombinase such as CreER^{T}, which is under the control of an endogenous glial cell-specific promoter, such as PLP. The animal further comprises a second heterologous sequence encoding a toxin such as DTA, which is downstream of a floxed region comprises a transcription termination signal (e.g. stop codon). The animal expresses CreER^{T} in oligodendrocytes, however, without being bound by theory, the CreER^{T} is unable to translocate into the nucleus and perform recombination. As a result, the animal does not express DTA. However, upon treatment with tamoxifen, CreER^{T} is activated and the floxed region comprising the transcription termination signal is removed and DTA can be transcribed and expressed, which induces cell death in the cells that express the CreER^{T}, such as oligodendrocytes.

Thus treatment with tamoxifen can be controlled temporally to regulate the timing of the ablation of the oligodendrocytes. The tamoxifen, or an analog thereof, can be administered to the animal at any stage of its development. The tamoxifen, or an analog thereof, can be administered once or more than once to the animal, at one or more stages of development. For example, the tamoxifen can be administered to the animal when the glial cells are mature, such as when the oligodendrocytes or Schwann cells are mature or non-proliferating. In some embodiments, the animal is a mouse, and the tamofixen, or an anlaog thereof, is administered when the mouse is at least approximately 5, 6, 7, 8, 9, 10, 11, or 12 weeks old. In some embodiments, the mice are treated with tamoxifen, or an analog thereof when the mice are between approximately 3 to 8 weeks old, approximately 5 to 7 weeks old, or approximately 6 to 8 weeks old. In some embodiments, the mice are treated with tamoxifen, or an analog thereof when the mice are at least approximately 6 months old. In yet other embodiments, the mice are treated with tamoxifen, or an analog thereof when the mice are at least approximately 7, 8, 9, 10, 11, 12, 15, 18, 21, or 24 months old. In some embodiments, the mice are treated with tamoxifen, or an analog thereof when the mice are between approximately 5 to 7 months old, 6 to 9 months old or 9 to 12 months old. In some embodiments, the animal is a mouse, and the tamofixen, or an analog thereof, is administered when the mouse is an adult mouse.

The ablation or cell death of glial cells, such as non-proliferating glial cells such as mature oligodendrocytes or Schwann cells in the non-human transgenic animals disclosed herein can result in demyelination in the animal. In some embodiments, the non-human transgenic animals remain viable. In some embodiments, the demyelination yields one or more phenotypic changes characteristic of a demyelination condition in the animal. In some embodiments, the one or more phenotypic changes characteristic of a demyelination condition in the animal are reversed. As described herein, a phenotype characteristic of a demyelination condition refers to a phenotype characteristic of a demyelination condition that is ascertainable without analysis of axons, myelinating cells, or other molecular or cellular analyses, such as electron microscopy of axons or determining gene expression of neural cells, such as oligodendrocytes or Schwann cells. For example, a phenotype change characteristic of a demyelination condition can be a decrease in motor control, balance, or CNS conduction, which can be measured by means such as a rotarod behavioral assay or spinal somatosensory evoked potential. Other phenotypes characteristic of a demyelination condition include phenotypes such as wobbly gait, hind limb paralysis, tremors, death, weight loss, ataxia, or any combination thereof. The phenotypes can be characteristic of a demyelination disorder is selected from the group consisting of Progressive Multifocal Leukoencephalopathy (PML), Encephalomyelitis, Central Pontine Myelolysis (CPM), Anti-MAG Disease, Leukodystrophies: Adrenoleukodystrophy (ALD), Alexander's Disease, Canavan Disease, Krabbe Disease, Metachromatic Leukodystrophy (MLD), Pelizaeus-Merzbacher Disease, Refsum Disease, Cockayne Syndrome, Van der Knapp Syndrome, and Zellweger Syndrome, Guillain-Barre Syndrome (GBS), chronic inflammatory demyelinating polyneuropathy (CIDP), multifocual motor neuropathy (MMN), Alzheimer's disease or progressive supernuclear palsy.

In some embodiments, the animal disclosed herein, such as a mouse, exhibits one or more phenotypic changes characteristic of a demyelination condition after an initial activation of the inducible Cre being expressed specifically in its glial cells, preferably mature or non-proliferating glial cells, such as oligodendrocytes, Schwann cells, or both. In some embodiments, the animal further exhibits a reversal of one or more of these phenotypic changes after the initial activation and exhibition of the one or more phenotypic changes. For example, in an animal that expresses an inducible Cre recombinase in myelinating cells and further comprises a heterologous sequence encoding DTA, wherein a floxed termination signal resides upstream of the sequence encoding DTA, the animal is treated with tamoxifen, which induces the myelinating cells undergo cell death. The animal exhibits demyelination, such as myelin sheath degeneration and vacuolation in distinct white-matter rich areas. Furthermore, the animal exhibits one or more phenotypic changes characteristic of a demyelination condition, such as decrease in motor control, decrease in balance, decrease in CNS conduction, wobbly gait, hind limb paralysis, tremors, weight loss, ataxia, or any combination thereof, which can be compared to control animals, such as wild type animals or animals with the same genetic background but not administered or treated with tamoxifen. After a given time period, the animal displays a reversal of the one or more phenotypes, such as an increase in motor control, increase in balance, improvement of CNS conduction, decrease in wobbly gait, decrease in hind limb paralysis, decrease in tremors, weight gain, decrease in ataxia, or any combination thereof.

In some embodiments, the reversal can be a complete reversal, wherein the animal recovers from the one or more phenotypic changes characteristic of a demyelination condition such that the animal exhibits phenotypes that a wild-type animal or an animal with the same genetic background but not induced to exhibit selective ablation of non-proliferating glial cells exhibits. In other embodiments, a reversal of a phenotype is when the phenotype improves. For example, at timepoint A, an animal begins to show tremors; at timepoint B, the animal has increased tremors; and at timepoint C, the animal shows a phenotype similar to as in timepoint A, which is an improved phenotype as compared to timepoint C. In another example, a reversal of a phenotype may be when there is a complete loss of a function, such as motor function or CNS conduction, and reversal is a gain in function, such as gain of motor function or CNS conduction. The gain can be a slight improvement in function. In some embodiments, the reversal can be regaining complete function, such as the to the same level of function as a wild-type animal or an animal with the same genetic background but not induced to exhibit selective ablation of non-proliferating glial cells.

In some embodiments, the animal displays a reversal of the one or more phenotypes characteristic of a demyelination disorder about 14 days or more, about 21 days or more, about 35 days or more, about 40 days or more, about 42 days or more, about 45 days or more, about 48 days or more, about 50 days or more, about 55 days or more, about 60 days or more, about 65 days or more, about 70 days or more, about 75 days or more, about 80 days or more, or about 90 days or more after an initial activation of the first heterologous protein, such as after activation of the inducible Cre recombinase in the animal or after administration of the exogenous agent that activates the inducible Cre recombinase. In some embodiments, the animal displays a reversal of the one or more phenotypes characteristic of a demyelination disorder about 35 days or more or about 70 days or more after an initial activation of the first heterologous protein, such as after activation of the inducible Cre recombinase. In other embodiments, the reversal begins about 2, 3, 4, 5, 6, 7, 8, 9, 10, 11, 12, 13, 14, 15, or 16 weeks after an initial activation of the first heterologous protein, such as after activation of the inducible Cre recombinase in the animal or after administration of the exogenous agent that activates the inducible Cre recombinase. In other embodiments, the reversal begins between about 2 to 12 weeks, about 3 to 12 weeks, about 4 to 12 weeks, about 5 to 12 weeks, about 5 to 11 weeks, or about 6 to 11 weeks after an initial activation of the first heterologous protein, such as after activation of the inducible Cre recombinase in the animal or after administration of the exogenous agent that activates the inducible Cre recombinase. In some embodiments, the reversal can be a complete reversal, wherein the animal recovers from the one or more phenotypic changes characteristic of a demyelination condition such that the animal exhibits phenotypes that a wild-type animal or an animal with the same genetic background but not induced to exhibit selective ablation of non-proliferating glial cells exhibits. In other embodiments, a reversal of a phenotype is when the phenotype improves.

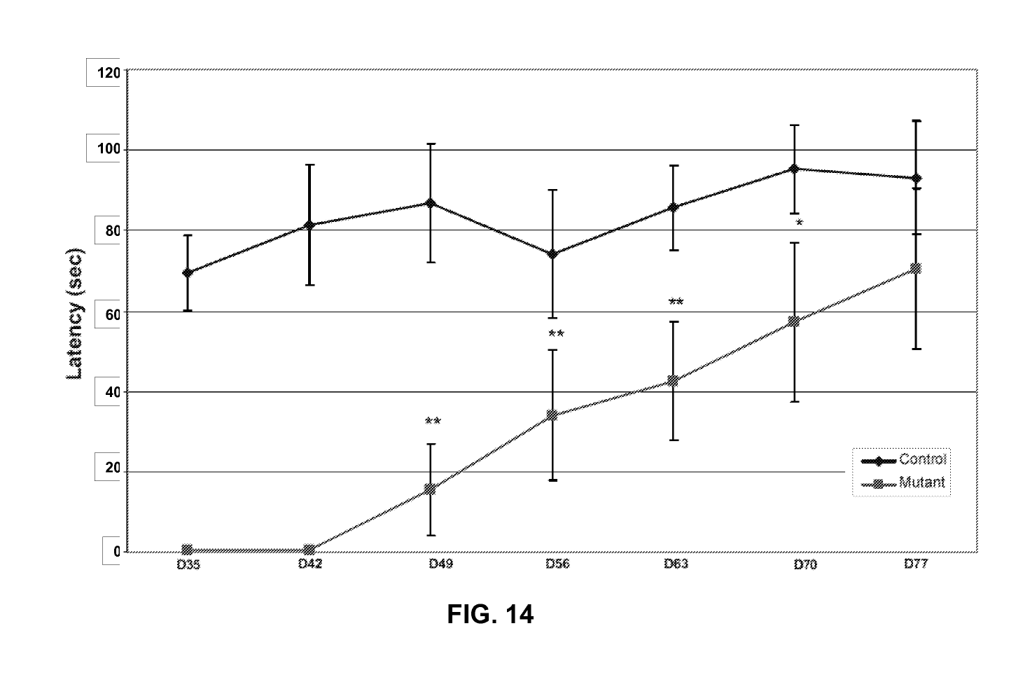

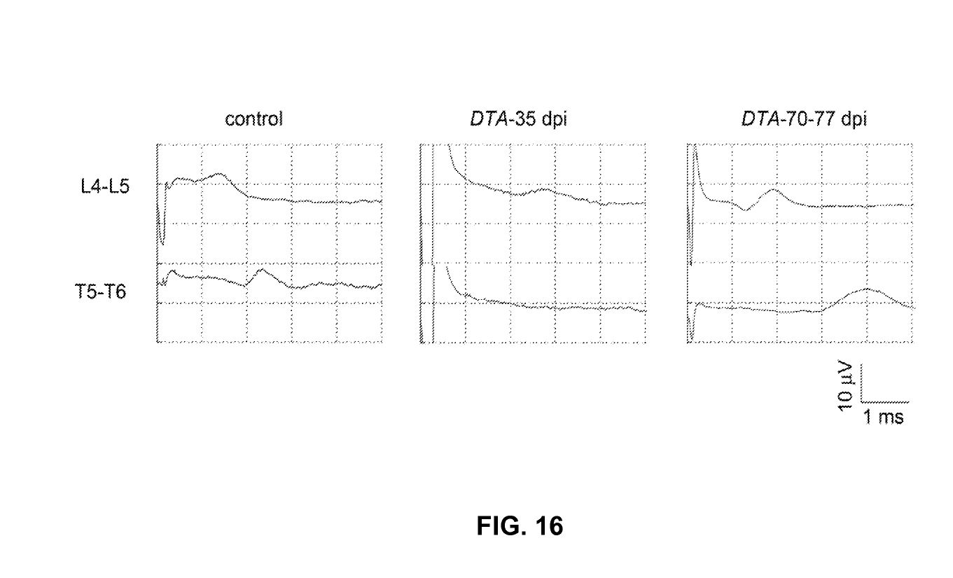

In one embodiment, a mouse exhibiting one or more one or more phenotypes characteristic of a demyelination disorder, such as decreased balance or motor control based on a rotarod assay, displays a reversal in the phenotype at about 42 days after administration of tamoxifen (see for example, FIG. 14), wherein the reversal is an improvement from essentially no motor control to gain of some motor control, though not yet to that of a control animal. In yet another embodiment, the mouse exhibits one or more one or more phenotypes characteristic of a demyelination disorder, such as decreased CNS conduction such as determined by sensory evoked potential, displays a reversal in the phenotype at about 77 days after administration of tamoxifen (see for example, FIG. 16), wherein the reversal is an improvement from essentially no CNS conduction to having CNS conduction, though not equal to that of a control animal.

The non-human transgenic animals disclosed herein can be treated with an exogenous agent that activates the first heterologous protein more than once. For example, a transgenic animal, preferably a mouse, comprising a first heterologous sequence encoding an inducible Cre recombinase such as CreER^{T} and a second heterologous sequence encoding a toxin such as DTA, which is downstream of a floxed region comprising a transcription termination signal, can be given a first treatment of tamoxifen or an analog thereof, followed by one, two, three, four, five or more additional treatments of tamoxifen. In some embodiments, the subsequent administration with the exogenous agent is after the reversal of the one or more phenotypes characteristic of a demyelination disorder. For example, a mouse exhibiting one or more one or more phenotypes characteristic of a demyelination disorder after an initial treatment with tamoxifen is administered a second treatment of tamoxifen after the reversal of one or more phenotypes characteristic of a demyelination disorder.

One can envision multiple cycles of the administration of the exogenous agent to activate the heterologous protein, where a single cycle of i) administration of an exogenous agent, ii) exhibition of one or more phenotypes characteristic of a demyelination disorder, and iii) reversal of the one or more phenotypes can be followed by another complete cycle of i), ii), and iii), or by a partial cycle thereof, such as i) and ii), or i) only. For example, after a single cycle of administration of an exogenous agent, exhibition of one or more phenotypes characteristic of a demyelination disorder, reversal of the one or more phenotypes, a second administration of an exogenous agent, exhibition of one or more phenotypes characteristic of a demyelination disorder, and reversal of the one or more phenotypes, a third administration of the exogenous agent can be performed. The subsequent administration of the exogenous agent after the initial administration, such as the second, third, fourth, fifth, or more administration, can be of a higher, equal, or lower amount of the exogenous agent as compared to the initial, previous, or subsequent amount of the exogenous agent.

The subsequent administration of the exogenous agent that activates the first heterelogous protein (ie. inducible Cre recombinase) is about 14 days or more, about 21 days or more, about 35 days or more, about 40 days or more, about 42 days or more, about 45 days or more, about 48 days or more, about 50 days or more, about 55 days or more, about 60 days or more, about 65 days or more, about 70 days or more, about 75 days or more, about 80 days or more, or about 90 days or more after an initial administration of the exogenous agent, or initial activation of the first heterelogous protein (ie. inducible Cre recombinase). In some embodiments, the subsequent administration of the exogenous agent that activates the first heterelogous protein (ie. inducible Cre recombinase) is about 70 days after an initial activation of the first heterologous protein, such as after activation of the inducible Cre recombinase. In other embodiments, the subsequent administration of the exogenous agent that activates the first heterelogous protein (ie. inducible Cre recombinase) is about 2, 3, 4, 5, 6, 7, 8, 9, 10, 11, 12, 13, 14, 15, or 16 weeks after an initial activation of the first heterologous protein, such as after activation of the inducible Cre recombinase in the animal or after administration of the exogenous agent that activates the inducible Cre recombinase.

In yet other embodiments, non-human transgenic animals disclosed herein can be treated with an exogenous agent that induces activation of a first heterologous protein, such as inducible Cre recombinase, and can further be administered with an immune-response inducing, or inflammation inducing agent such as an endotoxin. In some embodiments, the non-human transgenic animal is administered an exogenous agent that induces activation of a heterologous protein and an endotoxin such as lipopolysaccharides (LPS). The LPS can be administered prior to, concurrent with, or subsequent to, the administration of the exogenous agent that activates the heterologous protein (ie. tamoxifen or an analog thereof). More than one administration of LPS is also contemplated herein. For example, for each administration of tamoxifen, an administration of LPS is also given to the non-human transgenic animal.

In some embodiments, a transgenic animal, preferably a mouse, comprising a first heterologous sequence encoding an inducible Cre recombinase such as CreER^{T} and a second heterologous sequence encoding a toxin such as DTA, which is downstream of a floxed region comprising a transcription termination signal, can be given a first treatment of tamoxifen or an analog thereof, followed by administration of LPS. In some embodiments, the subsequent administration with LPS is prior to animal exhibiting one or more phenotypes characteristic of a demyelination disorder. For example, the LPS can be administered about 7 days or less, about 14 days or less, about 21 days or less, about 35 days or less, about 40 days or less, about 42 days or less, about 45 days or less, about 48 days or less, about 50 days or less, about 55 days or less, about 60 days or less, about 65 days or less, about 70 days or less, about 75 days or less, about 80 days or less, or about 90 days or less after an initial activation of the first heterelogous protein, such as after activation of the inducible Cre recombinase in the animal or after administration of the exogenous agent that activates the inducible Cre recombinase. In other embodiments, the administration of LPS is about 2, 3, 4, 5, 6, 7, 8, 9, 10, 11, 12, 13, 14, 15, or 16 weeks after an initial activation of the first heterologous protein, such as after activation of the inducible Cre recombinase in the animal or after administration of the exogenous agent that activates the inducible Cre recombinase.

The exogenous agent for inducing activation of a first heterologous protein in a non-human transgenic animal, such as tamoxifen or an analog thereof for the activation of inducible Cre recombinsase, can be administered by any means known in the art including, but not limited to, oral or parenteral routes, including intravenous, intramuscular, intraperitoneal, subcutaneous, transdermal, and airway (aerosol) administration. In some embodiments, more than one dose of the exogenous agent for inducing activation of a first heterologous protein is administered to the non-human transgenic animal disclosed herein. The one or more doses can be administered by the same or different means.

In addition to administration of the exogenous agent for inducing activation of a first heterologous protein, one or more doses of an immune-response inducing agent, such as LPS, can be administered. The immune-response inducing agent, such as LPS, can be administered by any means known in the art including, but not limited to, oral or parenteral routes, including intravenous, intramuscular, intraperitoneal, subcutaneous, transdermal, and airway (aerosol) administration. The administration of the immune-response inducing agent, such as LPS, can be administered by the same or different means as administration of the exogenous agent for inducing activation of a first heterologous protein (ie. tamoxifen or an analog thereof).

In some embodiments, more than one dose of immune-response inducing agent, such as LPS, can be administered to the non-human transgenic animal disclosed herein. The one or more doses can be administered by the same or different means used for administering the other doses of LPS. The exogenous agent, such as tamoxifen or an analog thereof, can be administered to a non-human transgenic animal together with the immune-response inducing agent, such as LPS, in a single composition. Alternatively, they can be administered substantially simultaneously, sequentially, at preset intervals throughout the day or treatment period, at different frequencies, or using the same or different routes of administration. The exogenous agent, such as tamoxifen or an analog thereof, and the immune-response inducing agent, such as LPS, can be administered to the non-human transgenic animal by the same or different means.

The exogenous agent for inducing activation of a first heterologous protein in a non-human transgenic animal, such as tamoxifen or an analog thereof for the activation of inducible Cre recombinase, can be administered focally or systemically. The immune-response inducing agent, such as LPS, can also be administered focally or systemically. Focal administration can be to the CNS, PNS, or the specific regions of the CNS or PNS. For example, the administration can be specifically to the brain, spinal cord, or optic nerve.

In some embodiments, the animal administered an immune-response inducing agent, such as LPS, can also have the expression of inflammation markers, such as, but not limited to, granulocyte/neturophil antigen 4/7, T cell antigen CD3, IBA1, and CD11b determined.

### Screening Assays

The non-human transgenic animals and methods disclosed herein provide a specific utility. The non-human transgenic animals and methods disclosed herein are useful for elucidating mechanisms of remyelination, as well as development of therapeutic strategies for promoting remyelination. This provides a real world application towards study of human diseases relating to demyelinating conditions, such as those described previously. Also, compared to other multiple sclerosis animal models, the mice described herein are more applicable to target validation during drug development, as the demyelination and remyelination events are highly reproducible, they occur at distinct timepoints in discrete locations, and are both clearly associated with a quantitative behavioral readout. Furthermore, this model system should allow for the exploration of factors that contribute to age-related decline in remyelination potential, such is in the diseases previously listed where subjects suffer from demyelination conditions. Lastly, the methods described herein for screening agents modulating myelination may be applied to identifying both enhancers and inhibitors of myelination, as both may be valuable targets for developing therapeutics for myelination disorders.

In one aspect, the non-human transgenic animal is used to screen candidate agents for an agent, or biologically active agent, that promotes remyelination or inhibit demyelination. In some embodiments, the method of selecting an agent comprises activating the first heterologous protein, such as inducible Cre recombinase, in the non-human transgenic animal, administering a candidate agent to said animal, determining one or more phenotypic changes characteristic of a demyelination condition in the animal; and, selecting the agent when the one or more phenotypic changes is reversed more quickly as compared to a control animal not administered the candidate agent. In other embodiments, the method of selecting an agent that promotes remyelination comprises activating the first heterologous protein, such as inducible Cre recombinase, in a non-human transgenic animal disclosed herein, administering a candidate agent to the animal, determining remyelination in the animal; and selecting the agent when the animal displays increased remyelination as compared to a control non-transgenic animal not administered the candidate agent.

In one aspect, the non-human transgenic animal is capable of being induced to exhibit selective ablation of non-proliferating glial cells or proliferating glial cells. In some embodiments, the animal is induced to specifically ablate non-proliferating glial cells. The induction of glial cell ablation can result in demyelination in the animal and the animal can exhibits one or more phenotypic changes characteristic of a demyelination condition, wherein the one or more phenotypic changes is reversed after the initial induction.

In some embodiments, the animal is a mouse, which comprises within its genome a first heterologous sequence encoding an inducible Cre recombinase, such as CreER^{T} or CreER^{T2}, operably linked to a promoter or regulatory region specific to neural cells, such as oligodendrocyte or Schwann cell specific promoters. The mouse also comprises a second heterologous sequence that encodes DTA, wherein DTA is expressed when the inducible Cre recombinase is activated by an exogenous agent, such as tamoxifen or an analog thereof. The expression of DTA can then induce cell death. Expression of DTA can be cell specific, such as specifically in neural cells, such as glial cells due to the expression of the Cre recombinase specifically in neural cells. Thus, expression of DTA can induce the cell-specific death, or cell-specific ablation, of oligodendrocytes, Schwann cells, or astrocytes. In some embodiments, the glial cells are non-proliferating glial cells, such as in the CNS, PNS, or both, wherein death of the non-proliferating glial cell results in demyelination in the CNS, PNS, or both. In some embodiments, the induction of inducible Cre recombinase results in mature oligodendrocyte cell death.

The administration of a candidate agent can be prior to, concurrent with, or after induction of the inducible Cre recombinase, such administration of the exogenous agent that activates the Cre recombinase (ie. tamoxifen or an analog thereof for the activation of CreER^{T} or CreER^{T2}). In some embodiments, the candidate agent is administered to the animal after induction of the inducible Cre recombinase, such as after administration of the exogenous agent that activates the Cre recombinase (ie. tamoxifen or an analog thereof for the activation of CreER^{T} or CreER^{T2}).

The candidate agent can be administered by any means known in the art including, but not limited to, oral or parenteral routes, including intravenous, intramuscular, intraperitoneal, subcutaneous, transdermal, and airway (aerosol) administration. In some embodiments, more than one dose of the candidate agent is administered to the non-human transgenic animal disclosed herein. In other embodiments, one or more candidate agents can be administered. The one or more doses, or one or more candidate agents, can be administered by the same or different means. Furthermore, the animal can also be administered one or more doses of the exogenous agent. In addition to administration of the one or more doses of the exogenous agent for inducing activation of a first heterologous protein, one or more doses of an immune-response inducing agent, such as LPS, can also be administered to the animal prior to, concurrent with, or subsequent to administration of the candidate agent. The candidate agent can be administered to a non-human transgenic animal together with the exogenous agent that activates inducible Cre recombinase (ie. as tamoxifen or an analog thereof), and optionally, the immune-response inducing agent (ie. LPS), in a single composition. Alternatively, the candidate agent can be administered substantially simultaneously, sequentially, at preset intervals throughout the day or treatment period, at different frequencies, or using the same or different routes of administration as the exogenous agent that activates inducible Cre recombinase, and optionally, LPS. The candidate agent, as well as the exogenous agent, and optionally, LPS, can be administered focally or systemically. Focal administration can be to the CNS, PNS, or the specific regions of the CNS or PNS. For example, the administration can be specifically to the brain, spinal cord, or optic nerve.

In some embodiments, the administration of a candidate agent, such as a single or multiple doses, can be prior to, concurrent with, or after the exhibition of the one or more phenotypic changes characteristic of a demyelination conditions, such as, but not limited to, wobbly gait, hind limb paralysis, tremors, decreased motor control, decrease balance, decreased CNS conduction, or any combination thereof. An agent can then be selected when the one or more phenotypic changes is reversed more quickly as compared to a control animal not administered the candidate agent. For example, in an animal that exhibits reversal of the one or more phenotypes characteristic of a demyelination disorder about 35 days or more after the activation of the Cre recombinase, or administration of an exogenous agent that activates the inducible Cre recombinase, an agent can be selected if an animal administered the candidate agent displays a reversal of the one or more phenotypes characteristic of a demyelination disorder in less than about 35 days.

In other embodiments, in an animal that exhibits reversal of the one or more phenotypes characteristic of a demyelination disorder about 70 days or more after the activation of the Cre recombinase, or administration of an exogenous agent that activates the inducible Cre recombinase, an agent can be selected if an animal administered the candidate agent displays reversal of the one or more phenotypes characteristic of a demyelination disorder in less than about 70 days. For example, a mouse administered tamoxifen to activate the inducible Cre recombinase expressed in mature oligodendroctyes exhibits decreased latency on a rotarod assay (see for example, FIG. 14) and start recovering after day 42 post administration of tamoxifen. In comparison, a mouse administered a candidate agent starts recovery in about 42 days or less post administration of tamoxifen. In some embodiments, the agent is selected when the reversal of the one or more phenotypes characteristic of a demyelination disorder in an animal administered the agent is about ½^{nd}, ⅓^{rd}, ¼^{th}, ⅕^{th}, ⅙^{th}, 1/7^{th}, ⅛^{th}, 1/9^{th}, 1/10^{th}, 1/11^{th}, 1/12^{th}, 1/14^{th}, 1/21^{st}, 1/28^{th}, 1/35^{th}, 1/42^{nd}, 1/49^{th}, 1/56^{th}, 1/63^{rd}, 1/70^{th}, or 1/77^{th} or less than the time for the reversal of the one or more phenotypes characteristic of a demyelination disorder in an animal not administered the agent. In some embodiments, detection and analysis (such as, but not limited to, video taping, weight measurements, rotarod assays, SEP assays, visual observations) is made at various time points and administration of a test agent can be repeated during the course of the assay, as well as using different dosing regimens.

In some embodiments, an agent is selected with the reversal of the one or more phenotypes characteristic of a demyelination disorder in an animal is faster or more quick than in an animal not administered the agent. The control animal not administered the candidate agent typically has the same genetic background as the animal administered the candidate agent, and also has its first heterologous protein (ie. inducible Cre recombinase) activated. In other embodiments, an agent is selected when the animal administered the candidate agent exhibits to a lesser degree (such as a lesser severity, lesser extent, or no exhibition) the one or more phenotypic changes characteristic of a demyelination condition as compared to a control animal not administered the candidate agent, wherein both animals have activated inducible Cre recombinase. In other embodiments, an agent is selected when the animal administered the candidate agent has a delay in the exhibition of one or more phenotypic changes characteristic of a demyelination condition as compared to a control animal not administered the candidate agent.

In yet other embodiments, an agent is selected when the animal administered the agent displays increased remyelination as compared to a control non-transgenic animal not administered the agent. For example, after activation of the inducible Cre recombinase in non-proliferating myelinating cells in a non-human transgenic animal disclosed herein, the animal expresses DTA in the cells, which induces cell death, resulting in demyelination in the animal. The demyelination condition can be characterized by a decrease in myelinated axons in the nervous systems (e.g., the central or peripheral nervous system), or by a reduction in the levels of markers of myelinating cells, such as oligodendrocytes and Schwann cells. Morphologically, neuronal demyelination can be characterized by a loss of oligodendrocytes in the central nervous system or Schwann cells in the peripheral nervous system. It can also be determined by a decrease in myelinated axons in the nervous system, or by a reduction in the levels of oligodendrocyte or Schwann cell markers. Exemplary marker proteins of oligodendrocytes or Schwann cells include, but are not limited to, CC1, myelin basic protein (MBP), ceramide galactosyltransferase (CGT), myelin associated glycoprotein (MAG), myelin oligodendrocyte glycoprotein (MOG), oligodendrocyte-myelin glycoprotein (OMG), cyclic nucleotide phosphodiesterase (CNP), NOGO, myelin protein zero (MPZ), peripheral myelin protein 22 (PMP22), protein 2 (P2), galactocerebroside (GalC), sulfatide and proteolipid protein (PLP). As such, the candidate agents identified by the subject method encompass substances that can inhibit the deleterious morphological characteristics of neuronal demyelination.

Thus, in some embodiments, the remyelination is characterized by myelinated axons. For example, myelinated axons can be detected using any means known in the arts, such as by electron microscopy. In other embodiments, remyelination can be characterized by expression of markers for astrocytes or microglia cells, for example, decreased expression of microglial cell markers representing decreased microglial cells. In some embodiments, remyelination is characterized by the expression of one or more oligodendrocyte cell markers. For example, the expression of one or more remyelination-specific marker proteins of an animal treated with a candidate agent can be compared to a control or reference animal. A wide variety of labels suitable for detecting protein levels are known in the art. Non-limiting examples include radioisotopes, enzymes, colloidal metals, fluorescent compounds, bioluminescent compounds, and chemiluminescent compounds. In other embodiments, the RNA level or mRNA level of the marker is detected, such as by PCR, RT-PCR, or other means known in the arts. The oligodendrocyte cell marker can be selected from the group consisting of, but not limited to, CC1, myelin basic protein (MBP), ceramide galactosyltransferase (CGT), myelin associated glycoprotein (MAG), myelin oligodendrocyte glycoprotein (MOG), oligodendrocyte-myelin glycoprotein (OMG), cyclic nucleotide phosphodiesterase (CNP), NOGO, myelin protein zero (MPZ), peripheral myelin protein 22 (PMP22), protein 2 (P2), galactocerebroside (GalC), sulfatide and proteolipid protein (PLP).

In yet other embodiments, the expression of remyelination-specific marker proteins, such as measuring protein level or mRNA level, is measured in the test animal and a control or reference animal, in determining whether a candidate agent has remyelination inhibiting or reducing activity. Such an agent can be categorized as a remyelination inhibitor or remyelination toxin.

As disclosed herein, remyelination can be ascertained by observing an increase in the cell-specific expression of a marker gene/gene product (e.g., in the central or peripheral nervous system). Fluorescence of the marker proteins can be detected using in vitro or in vivo methods known in the art for detection of fluorescence in small animals. In vivo fluorescence can be detected and/or quantified utilizing devices available in the relevant art. Visualization, imaging or detection can be made through invasive, minimally invasive or non-invasive techniques. Typically, microscopy techniques are utilized to detect or image fluorescence from cells/tissue obtained from the transgenic animals, from living cells, or through in vivo imaging techniques.

Luminescent, fluorescent or bioluminescent signals are easily detected and quantified with any one of a variety of automated and/or high-throughput instrumentation systems including fluorescence multi-well plate readers, fluorescence activated cell sorters (FACS) and automated cell-based imaging systems that provide spatial resolution of the signal. A variety of instrumentation systems have been developed to automate HCS (high-content screening) including the automated fluorescence imaging and automated microscopy systems developed by Cellomics, Amersham, TTP, Q3DM, Evotec, Universal Imaging and Zeiss. Fluorescence recovery after photobleaching (FRAP) and time lapse fluorescence microscopy have also been used to study protein mobility in living cells.

Visualizing fluorescence (e.g., marker gene encoding a fluorescent protein) can be conducted with microscopy techniques, either through examining cell/tissue samples obtained from an animal (e.g., through sectioning and imaging using a confocal microscope), examining living cells or detection of fluorescence in vivo. Visualization techniques include but are not limited utilization of confocal microscopy or photo-optical scanning techniques known in the art. Generally, fluorescence labels with emission wavelengths in the near-infrared are more amenable to deep-tissue imaging because both scattering and autofluorescence, which increase background noise, are reduced as wavelengths are increase. Examples of in vivo imaging are known in the art, such as disclosed by Mansfield et al., J. Biomed. Opt. 10:41207 (2005); Zhang et al., Drug Met. Disp. 31:1054-1064 (2003); Flusberg et al., Nat. Meth. 2:941-950 (2005); Mehta et al., Curr Opin Neurobiol. 14:617-628 (2004); Jung et al., J. Neurophysiol. 92:3121-3133 (2004); U.S. Pat. Nos. 6,977,733 and 6,839,586, each disclosure of which is herein incorporated by reference.

Moreover, demyelination/remyelination phenomena can be observed by immunohistochemical means or protein analysis known in the art. For example, sections of the test animal's brain can be stained with antibodies that specifically recognize an oligodendrocyte marker. In another aspect, the expression levels of oligodendrocyte markers can be quantified by immunoblotting, hybridization means, and amplification procedures, and any other methods that are well-established in the art. e.g., Mukouyama et al., Proc. Natl. Acad. Sci. 103:1551-1556 (2006); Zhang et al., supra; Girard et al., J. Neurosci. 25:7924-7933 (2005); and U.S. Pat. Nos. 6,909,031; 6,891,081; 6,903,244; 6,905,823; 6,781,029; and 6,753,456, the disclosure of each of which is herein incorporated by reference.

In another aspect, cell/tissue from the central or peripheral nervous system can be excised and processed for protein, e.g., tissue is homogenized and protein is separated on an SDS-10% polyacylamide gel and then transferred to nitrocellulose membrane to detect marker proteins. Fluorescent protein levels can be detected utilizing primary antibody/antisera (e.g., goat polyclonal raised against a particular marker protein; BD Gentest, Woburn, Mass.) and peroxidase-conjugated secondary antibody rabbit anti-goat IgG (Sigma-Aldrich). Chemiluminescence is detected using standard reagents available in the art to detect and determine levels of fluorescence marker proteins in tissue samples.

In another aspect of the present invention, the transgenic animals disclosed herein can be the source for cell/tissue culture. For example, a cell from an animal disclosed herein can be used for cell-based assays for providing a comparison of the expression of a gene or gene product or the activity of said gene product in a test neural cell (e.g., transgenic oligodendrocyte or Schwann cell) relative to a control cell, a cell obtained from a control animal. The cell can be a proliferating cell or a non-proliferating cell, such as a mature or differentiated oligodendrocyte or Schwann cell. The test neural cell can be isolated from the CNS or PNS, or cell culture derived from the cells of the transgenic animals, the progeny thereof, and section or smear prepared from the source, or any other samples of the brain that contain, for example, oligodendrocytes or Schwann cells or their progenitors. Where desired, one can choose to use enriched cell cultures that are substantially free of other neural cell types such as neurons, microglial cells, and astrocytes. Various methods of isolating, generating or maintaining matured oligodendrocytes and Schwann cells are known in the art (Baerwald et al., J. Neurosci. Res. 52:230-239 (1998); Levi et al., J Neurosci. Meth. 68:21-26 (1998)) and are exemplified herein.

Also provided herein is a candidate agent, or a biologically active agent that is selected, which can include, but not be limited to a biological or chemical compound such as a simple or complex organic or inorganic molecule, peptide, peptide mimetic, protein (e.g. antibody), liposome, small interfering RNA, or a polynucleotide (e.g. anti-sense). A vast array of compounds can be synthesized, for example polymers, such as polypeptides and polynucleotides, and synthetic organic compounds based on various core structures, and these are also contemplated herein. In addition, various natural sources can provide compounds for screening, such as plant or animal extracts, and the like. It should be understood, although not always explicitly stated that the active agent can be used alone or in combination with another modulator, having the same or different biological activity as the agents identified by the subject screening method.

A candidate agent, such as a therapeutic/drug, assayed in one or more methods disclosed herein, can be used in the model systems such as the transgenic animals disclosed herein to determined if there is an overall difference in response to the drug compared at different time points, as well as compared to reference or controls. By detecting and quantifying expression of a marker, the transgenic animal can be used to determine whether a candidate agent modulates remyelination and to what degree. Of course, one of ordinary skill in the art will recognize that the foregoing is merely one example for utilizing the model system disclosed herein.

Therefore, if a candidate therapeutic/drug is being assayed in one or more methods of the invention, then it can be determined if there is an overall difference in response to the drug compared at different time points, as well as compared to reference or controls. In summary, by detecting and quantifying expression of a marker which is differentially expressed in a single subpopulation of glial cells (e.g., progenitor oligodendrocytes) the transgenic animal in the foregoing example is used to obtain various data, which include whether remyelination is occurring post insult and whether a candidate agent modulates such remyelination and to what degree. Of course, one of ordinary skill in the art will recognize that the foregoing is merely one example for utilizing the remyelination model system of the present invention.

### Pharmaceutical Compositions

The one or more methods disclosed herein can be utilized to select a biologically active agent that can subsequently be implemented in treatment of demyelination, by inhibiting demyelination or promoting remyelination. The selected biologically active agents effective to modulate remyelination can be used for the preparation of medicaments for treating neuronal demyelination disorders. In certain embodiments, the demyelination disorder referred herein is multiple sclerosis. In other embodiments, the demyelination disorder is selected from the group consisting of Progressive Multifocal Leukoencephalopathy (PML), Encephalomyelitis, Central Pontine Myelolysis (CPM), Anti-MAG Disease, Leukodystrophies: Adrenoleukodystrophy (ALD), Alexander's Disease, Canavan Disease, Krabbe Disease, Metachromatic Leukodystrophy (MLD), Pelizaeus-Merzbacher Disease, Refsum Disease, Cockayne Syndrome, Van der Knapp Syndrome, and Zellweger Syndrome, Guillain-Barre Syndrome (GBS), chronic inflammatory demyelinating polyneuropathy (CIDP), and multifocual motor neuropathy (MMN).

In one aspect, an identified/selected biologically active agent of this invention can be administered to treat neuronal demyelination inflicted by pathogens such as bacteria and viruses. In another aspect, the selected agent can be used to treat neuronal demyelination caused by toxic substances or accumulation of toxic metabolites in the body as in, e.g., central pontine myelinolysis and vitamin deficiencies. In yet another aspect, the agent can be used to treat demyelination caused by physical injury, such as spinal cord injury. In still yet another aspect, the agent can be administered to treat demyelination manifested in disorders having genetic attributes, genetic disorders including but not limited to leukodystrophies, adrenoleukodystrophy, degenerative multi-system atrophy, Binswanger encephalopathy, tumors in the central nervous system, and multiple sclerosis.

The identified/selected biologically active agent of the invention can also be delivered with, prior to, or subsequent to, other products of interest that include, but not be limited to: a growth factor, cytokine, nerve growth factor, anti-sense RNA, siRNA, immuno-suppressants, anti-inflammatories, anti-proliferatives, anti-migratory agents, anti-fibrotic agents, pro-apoptotics, antibodies, anti-thrombotic agents, anti-platelet agents, IIbIIIIa agents, angiogenic factors, anti-angiogenic factors, antiviral agents, nerve growth factor, NGF family of proteins, NGF, Beta-NGF, Neurotrophin-3 precursor (NT-3), HDNF, Nerve growth factor 2 (NGF-2), Brain-derived neurotrophic factor (BDNF), Neurotrophin-5 (NT-5), Neurotrophin-4 (NT-4), or precursors and combinations thereof.

Various delivery systems are known and can be used to administer a biologically active agent, e.g., encapsulation in liposomes, microparticles, microcapsules, expression by recombinant cells, receptor-mediated endocytosis (see, e.g., Wu and Wu, J. Biol. Chem. 262:4429-4432 (1987)), construction of a therapeutic nucleic acid as part of a retroviral or other vector, etc. Methods of delivery include but are not limited to intra-arterial, intra-muscular, intravenous, intranasal, and oral routes. In a specific embodiment, it can be desirable to administer the pharmaceutical compositions of the invention locally to the area in need of treatment; this can be achieved by, for example, and not by way of limitation, local infusion during surgery, by injection, or by means of a catheter. In certain embodiment, the agents are delivered to a subject's nerve systems, preferably the central nervous system. In another embodiment, the agents are administered to neural tissues undergoing remyelination.

Administration of the selected agent can be effected in one dose, continuously, or intermittently throughout the course of treatment. Methods of determining the most effective means and dosage of administration are well known to those of skill in the art and will vary with the composition used for therapy, the purpose of the therapy, the target cell being treated, and the subject being treated. Single or multiple administrations can be carried out with the dose level and pattern being selected by the treating physician.

The preparation of pharmaceutical compositions of this invention is conducted in accordance with generally accepted procedures for the preparation of pharmaceutical preparations. See, for example, Remington's Pharmaceutical Sciences 18th Edition (1990), E.W. Martin ed., Mack Publishing Co., PA. Depending on the intended use and mode of administration, it can be desirable to process the active ingredient further in the preparation of pharmaceutical compositions. Appropriate processing can include mixing with appropriate non-toxic and non-interfering components, sterilizing, dividing into dose units, and enclosing in a delivery device.

Pharmaceutical compositions for oral, intranasal, or topical administration can be supplied in solid, semi-solid or liquid forms, including tablets, capsules, powders, liquids, and suspensions. Compositions for injection can be supplied as liquid solutions or suspensions, as emulsions, or as solid forms suitable for dissolution or suspension in liquid prior to injection. For administration via the respiratory tract, a some composition is one that provides a solid, powder, or aerosol when used with an appropriate aerosolizer device.

Liquid pharmaceutically acceptable compositions can, for example, be prepared by dissolving or dispersing a polypeptide embodied herein in a liquid excipient, such as water, saline, aqueous dextrose, glycerol, or ethanol. The composition can also contain other medicinal agents, pharmaceutical agents, adjuvants, carriers, and auxiliary substances such as wetting or emulsifying agents, and pH buffering agents.

### EXAMPLES

### Example 1

### Generation of PLP/CreER^{T}

### ROSA26-eGFP-DTA Mice and Induction of Cre Recombinase Activity

A mouse comprising the ROSA26-eGFP-DTA allele (Ivanova et al., Genesis 43:129-135 (2005)), which harbors the gene of the Diptheria toxin A chain (DT-A) in a functionally inactive form due to the presence of the upstream floxed DNA sequence region that contain eGFP and Neo genes, was crossed with a mouse with a PLP/CreER^{T} allele (Doerflinger et al., Genesis, 35:63-72 (2003)), which express the tamoxifen-related version of the Cre recombinase in oligodendrocytes. The mating resulted in compound heterozygous PLP/CreER^{T}; ROSA26-eGFP-DTA mice, in which Cre-mediated excision of the floxed region is induced with tamoxifen in the PLP/CreER^{T}; ROSA26-eGFP-DTA mouse, resulting in DTA expression (FIG. 1).

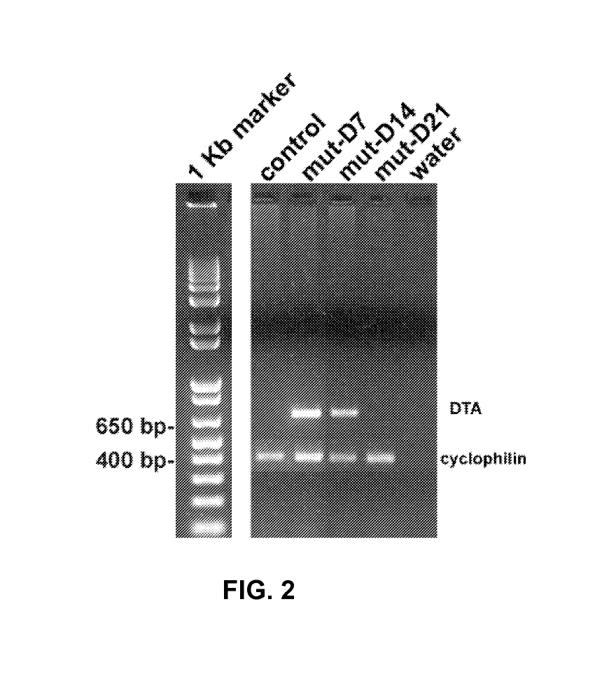

At 5-7 weeks old, the mice were administered 1 mg of tamoxifen a day for five to seven days, intraperitoneally (IP). The expression of the DTA in the brain of the tamoxifen treated mice was confirmed by PCR. The P1 primer (5′-AAACTCTTCGCGGTCTTTC-3′) and P2 primer (5′-CTTAACGCTTTCGCCTGTTC′3′) was used and the binding sites are depicted in FIG. 1. Upon recombination, the P1 and P2 primers amplify the approximately 650 bp (DTA) product as depicted in FIG. 2. The control lane depicts the PCR result from the ROSA26-eGFP-DTA mice without induced expression of DTA and the results from the tamoxifen-treated mice treated at 7, 14, and 21 days after the first tamoxifen injection (dpi) are depicted in lanes mut-D7, mut-D14, and mut-D21, respectively. Without being bound by theory, the eventual decrease in DTA expression can be attributed to the death, and lack of, cells.

### Example 2

### Demyelination and Phenotypic Characteristics of the Tamoxifen-Treated Mice

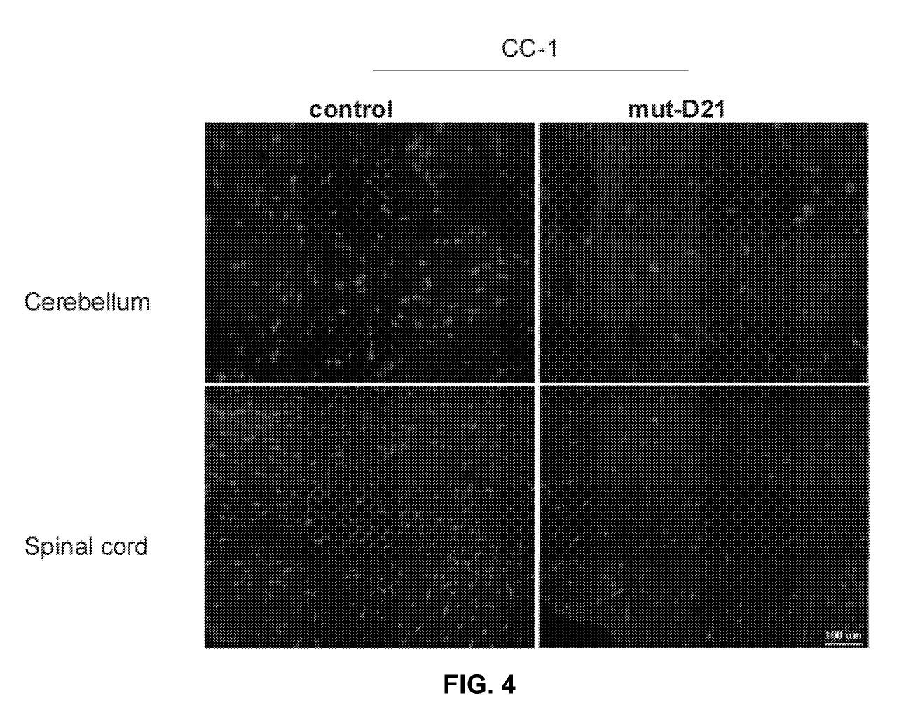

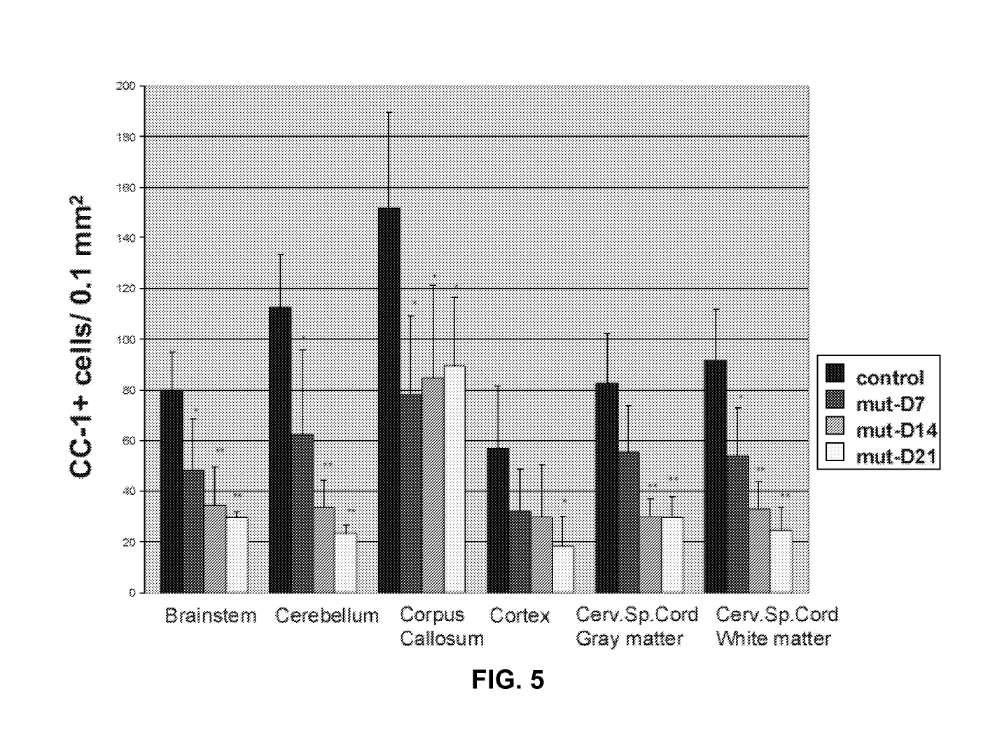

The tamoxifen treated PLP/CreER^{T}; ROSA26-eGFP-DTA mice were characterized to detect the loss of oligodendrocytes. To determine the oligodendrocyte cell loss, CC-1 staining in the brainstem, cerebellum, corpus callosum, cortex, cervical spinal cord (gray and white matter) was performed and quantitated in the tamoxifen-treated mice PLP/CreER^{T}; ROSA26-eGFP-DTA mice 7, 14, and 21 dpi (FIG. 5). Oligodendrocyte cell loss was a maximum in most CNS areas of the tamoxifen-treated mice PLP/CreER^{T}; ROSA26-eGFP-DTA mice by 21 dpi (see for example, FIG. 4, 5).

RT-PCR with the following primers was performed:

mouse PLP sense primer:
CACTTACAACTTCGCCGTCCT
mouse PLP anti-sense primer:
GGGAGTTTCTATGGGAGCTCAGA
mouse PLP probe:
AACTCATGGGCCGAGGCACCAA
mouse MBP sense primer:
GCTCCCTGCCCCAGAAGT
mouse MBP anti-sense primer:
TGTCACAATGTTCTTGAAGAAATGG
mouse MBP probe:
AGCACGGCCGGACCCAAGATG
mouse GAPDH sense primer:
CTCAACTACATGGTCTACATGTTCCA
mouse GAPDH anti-sense primer:
CCATTCTCGGCCTTGACTGT
mouse GAPDH probe:
TGACTCCACTCACGGCAAATTCAACG

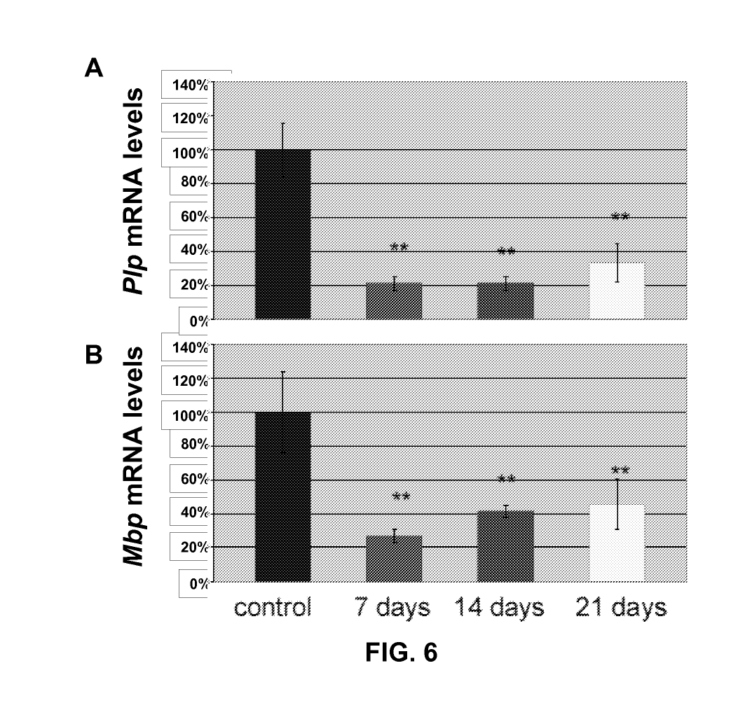

The results indicated a dramatic drop of Plp and Mbp mRNA levels that preceded oligodendrocyte cell loss in the tamoxifen-treated mice PLP/CreER^{T}; ROSA26-eGFP-DTA mice at 7, 14, or 21 dpi, as compared to control mice (FIG. 6).

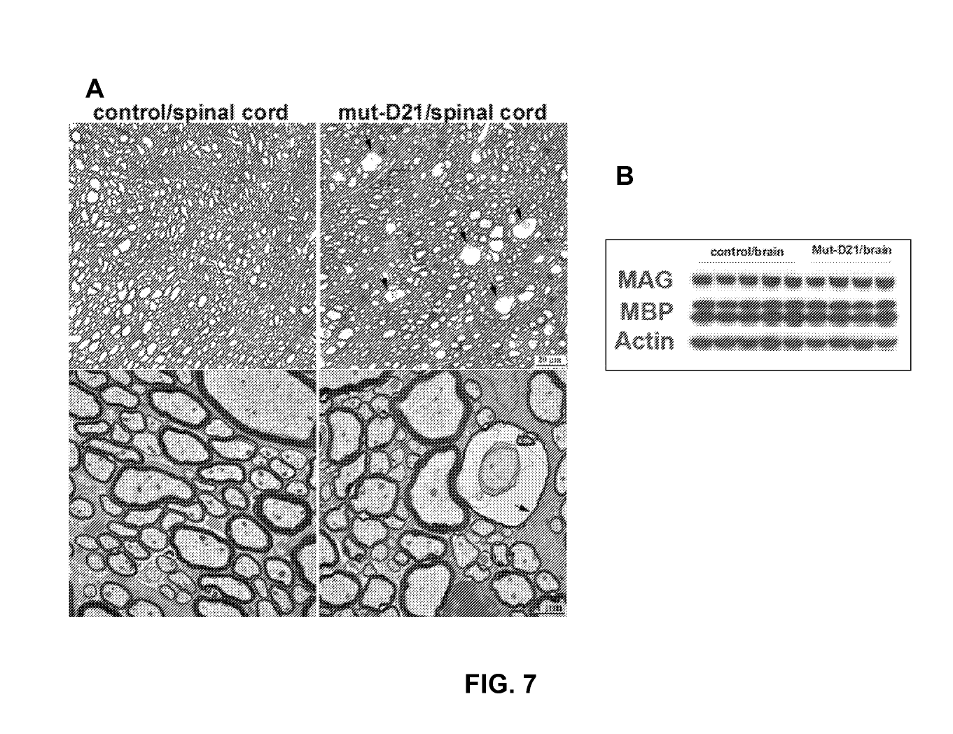

CNS myelination was determined by electron microscopy (EM) and toluidine blue staining of the spinal cord and Western blotting of MAG and MBP protein expression in the brain. The impact of oligodendrocyte cell loss was minimal in the tamoxifen-treated PLP/CreER^{T}; ROSA26-eGFP-DTA mice 21 dpi (FIG. 7). However, at 14 dpi, the mice displayed phenotypes characteristic of a demyelination condition. The mice exhibited tremors and an ataxic uncoordinated gait. By 21 dpi, some mice become very weak and died.

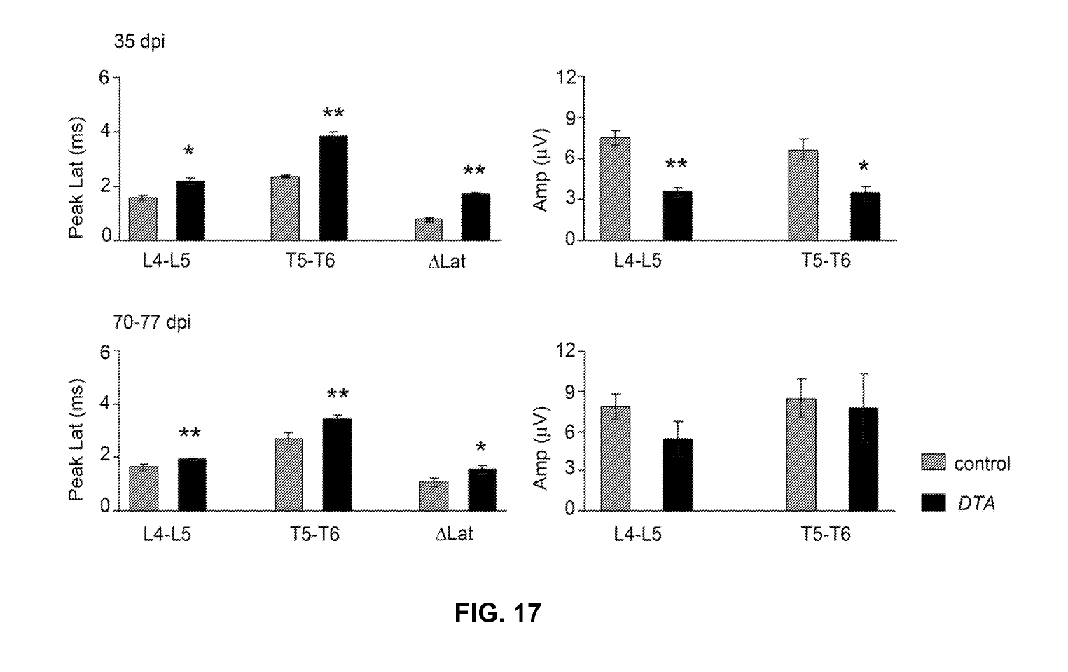

The tamoxifen-treated PLP/CreER^{T}; ROSA26-eGFP-DTA mice 21 dpi that survived, had no SEP response. SEP recording were possible in DTA mice at 35 and 70-77 dpi (FIG. 16 and FIG. 17). To determine the SEP response, control and tamoxifen-treated PLP/CreER^{T}; ROSA26-eGFP-DTA mice were anesthetized with intraperitoneal injections of Avertin®, and the stimulating electrodes were introduced subcutaneously in one of the three different sciatic nerve levels: (1) ankle, (2) popliteal fossa, or (3) directly at the roots. The ground electrode was applied to the tail upon stimulation of the nerve and recordings were made from the low lumbar or the mid-thoracic level. The different between the onset latency of the sensory evoked potential (SEP) recorded from the two levels (ΔL) was calculated as an estimate of the conduction in the CNS.

At 35 dpi and even 42 days PI, the tamoxifen-treated PLP/CreER^{T}; ROSA26-eGFP-DTA mice were unable to perform on the rotarod (FIG. 14). The mice were tested for the time (latency) they maintained themselves on the rod rotating at the accelerating speed mode (5 to 65 rpm) during a 5 minute trial session. The latency time was monitored for each mouse by performing 4 sessions a day, once a week, for 7 consecutive weeks.

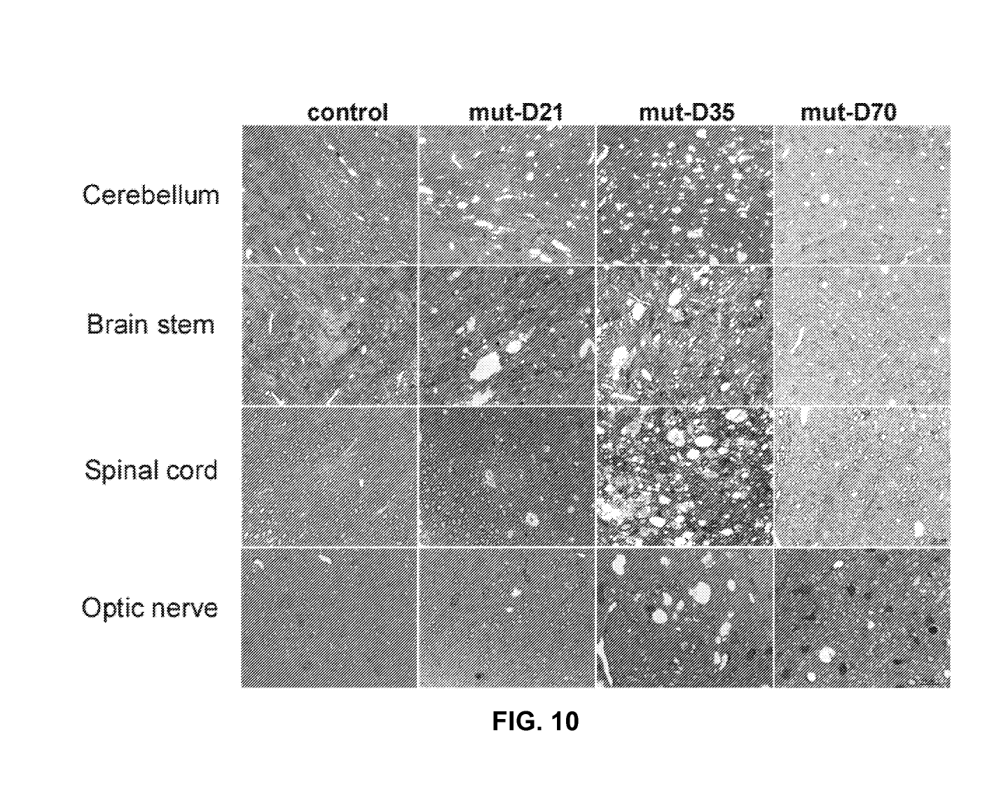

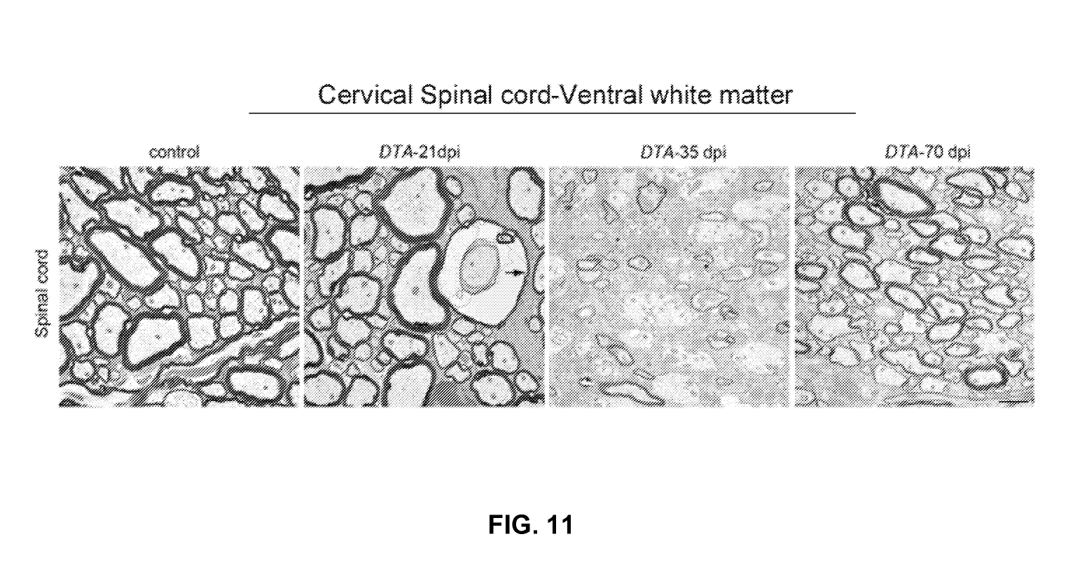

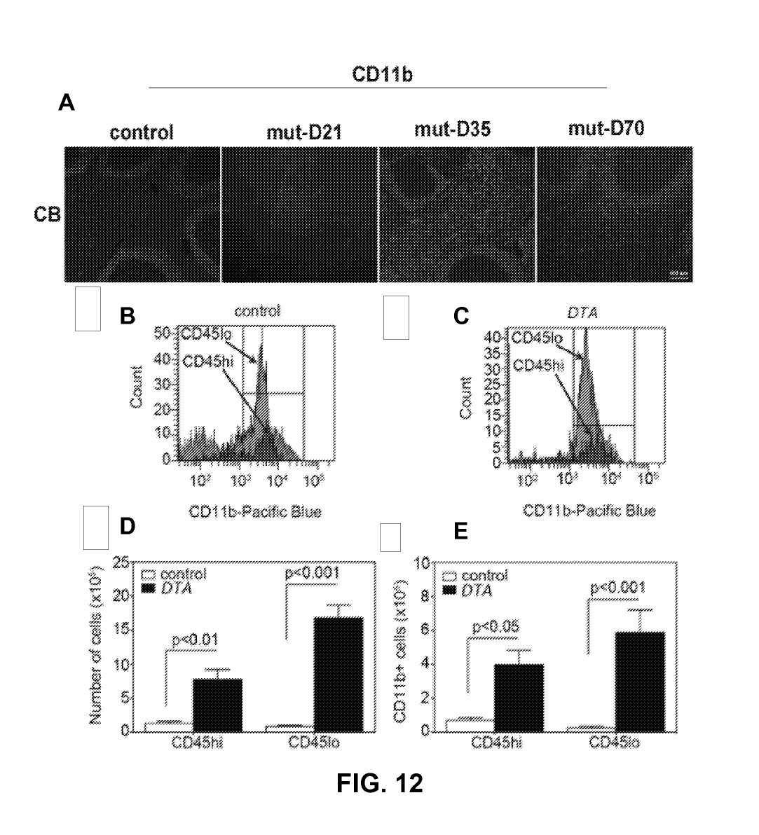

Also at 35 dpi, the mice showed increased ataxia and tremor, and toluidine blue staining of the cerebellum, brain stem, spinal cord, and optic nerve of the mice revealed massive oligodendrocyte loss throughout the CNS resulted in myelin sheath degeneration and vacuolation in distinct white matter-rich areas (FIG. 10). An EM of the cervical spinal cord-ventral white matter at 35 dpi also demonstrated demyelination (FIG. 11). Microglia activation, as determined by CD11b staining of microglia in the cerebellum, also correlated with demyelination at 35 dpi (FIG. 12).

### Example 3

### Remyelination and Reversal of Phenotypic Characteristics of Demyelination

Surprisingly, the tamoxifen treated PLP/CreER^{T}; ROSA26-eGFP-DTA mice were able to remyelinate and displayed reversal of the phenotype characteristics of demyelination. The animals showed a remarkable phenotypic recovery. As described in Example 2, the tamoxifen treated PLP/CreER^{T}; ROSA26-eGFP-DTA mice were unable to perform on the rotarod at 35 dpi and even 42 dpi. However, the mice started to regain their motor function after 42 dpi and by 77 dpi, the latency time of the tamoxifen treated PLP/CreER^{T}; ROSA26-eGFP-DTA mice was eventually approximately that of the control mice (FIG. 14). The tamoxifen-treated PLP/CreER^{T}; ROSA26-eGFP-DTA mice, which had no SEP response at 21 dpi, had an SEP response by 70 dpi, when Δlatency (ΔLat, difference between the T5-T6 and L4-L5 peak latencies) is slightly lower than controls and amplitude (Amp) is normal (FIG. 17).

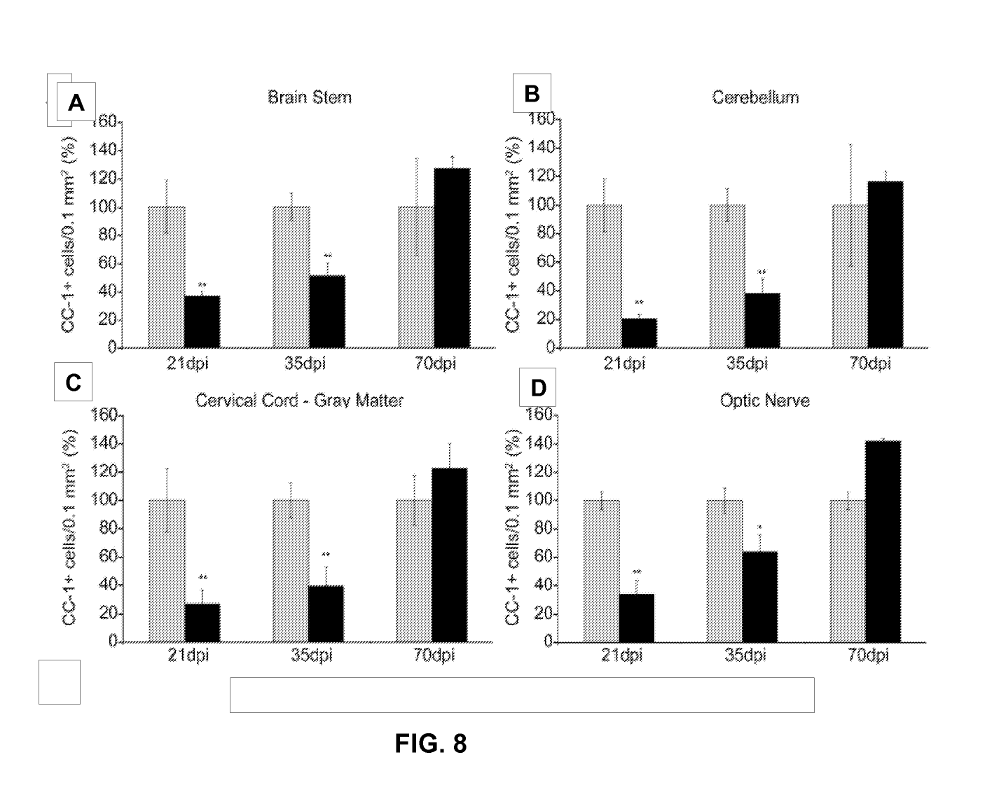

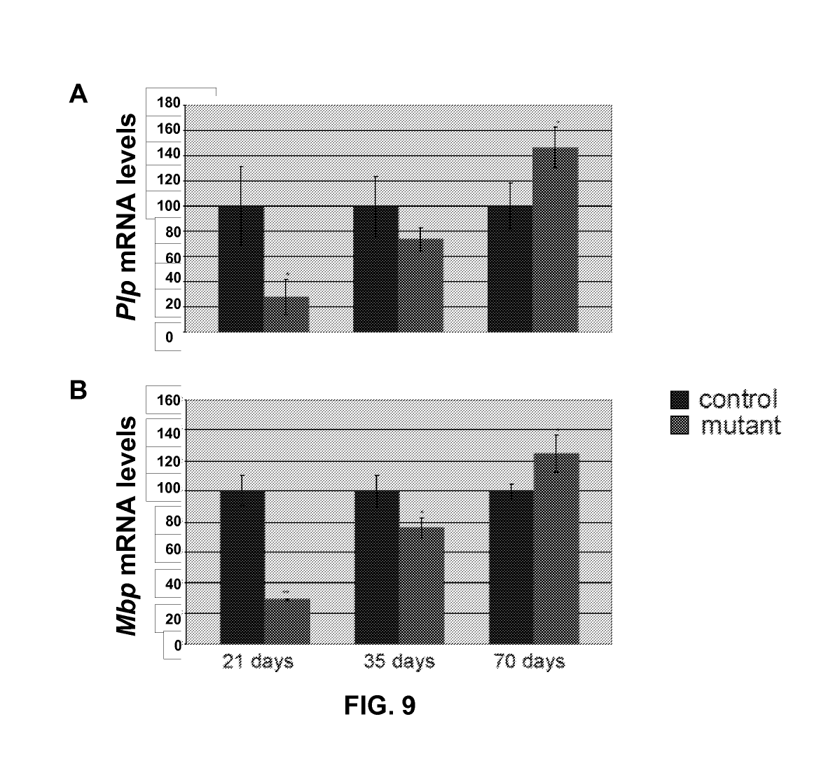

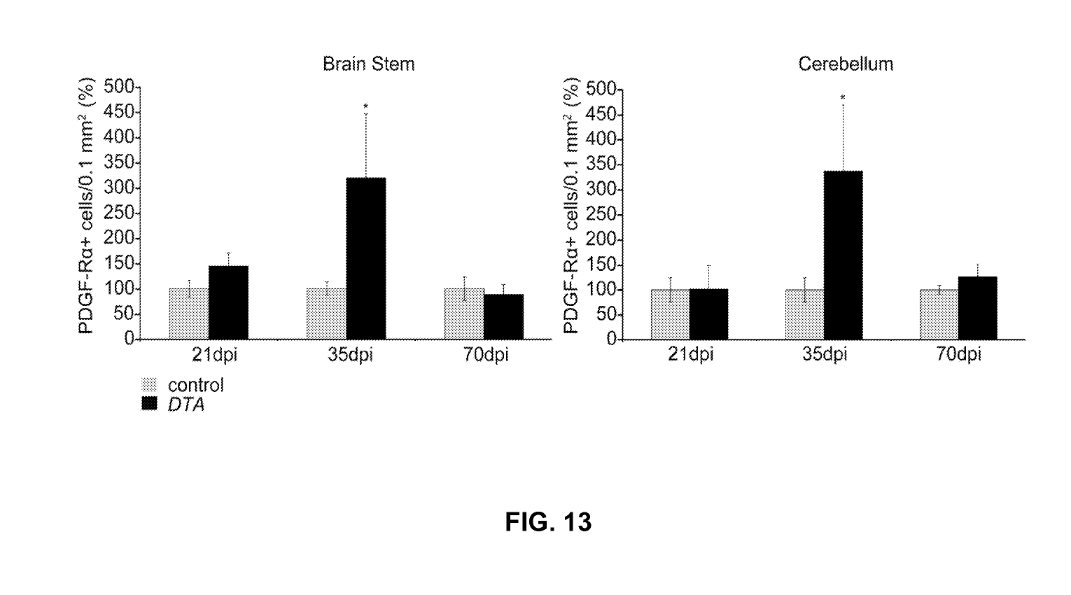

Unexpectedly, the tamoxifen-treated PLP/CreER^{T}; ROSA26-eGFP-DTA mice were replenishing and repairing the oligodendrocyte-depleted areas. The oligodendrocyte cell numbers were increasing by 35 dpi, with even more by 70 dpi, as indicated by CC1 immunostaining (FIG. 8). RT-PCR analysis of Mbp and Plp mRNA expression showed a similar trend, where by 70 dpi, the levels were even higher than control mice (FIG. 9). Toluidine blue staining of the cerebellum, brain stem, spinal cord, and optic nerve of the mice also revealed remyelination at 70 dpi (FIG. 10), as did EM of the cervical spinal cord-ventral white matter also indicated remyelination (FIG. 11). Microglia activation had also decreased, correlating with remyelination (FIG. 12). Furthermore, oligodendrocyte precursor cells (OPC), as measured by PDGFRα positive cells in the brain stem and cerebellum, was also increased as compared to the control mice (FIG. 13).

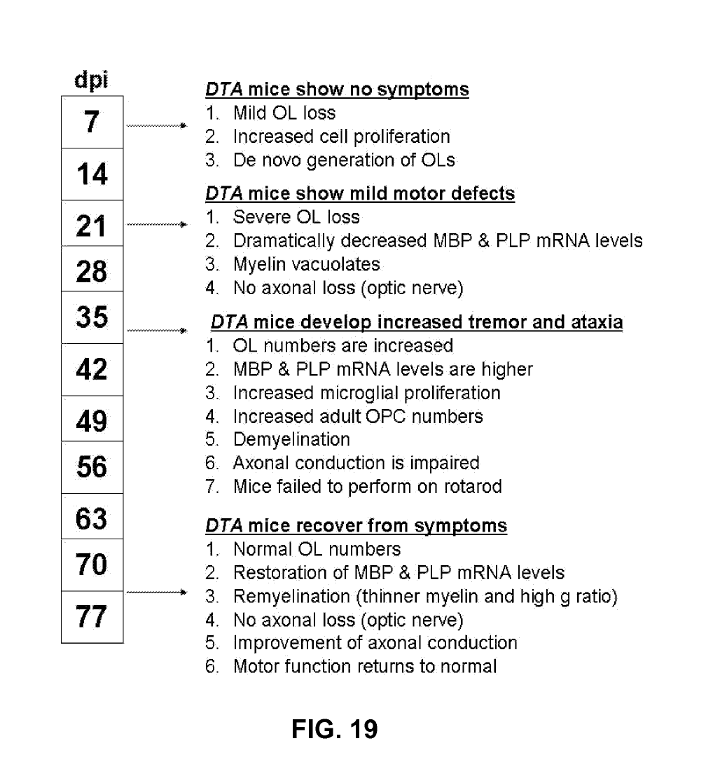

A summary of the demyelination and display of the phenotypes characteristic of a demyelination disorder, and the unexpected remyelination and reversal of the phenotypes is shown in FIG. 19.

### Example 4

### Titration of Tamoxifen in PLP/CreER^{T}

### ROSA26-eGFP-DTA Mice

PLP/CreER^{T}; ROSA26-eGFP-DTA mice are administered various amounts of tamoxifen or 4-hydroxytamoxifen (4-OHT) of various regimens to evaluate the range of demyelination and phenotypic changes characteristic of a demyelination disorder. Doses ranging from approximately 1-7 mg of 4-OHT is administered IP to different groups of mice. The dosing regimen, of approximately once a day for 3 to 7 sequential days, is also varied for different groups of mice. The different groups of mice also comprise younger mice (5-6 weeks of age) and older mice (6-9 months of age).

The mice are characterized by CC1 immunostaining, CD11b staining, EM, toluidine blue staining, Mbp and Plp gene expression, rotarod assay, analysis of the gait by the treadmill DigiGait assay, and SEP assay. Immunohistochemistry is also performed. The extent of phenotypic changes, such as lower severity of clinical symptoms, longer time to show clinical symptoms, or faster recovery time is expected with lower dosages or shorter times of administration as compared to the regimen described in Example 1. The mice with lower dosages and can also have lower percentage of mice dying. The effect and any differences, such as time for recovery or extent of recovery, of the younger versus older mice are also analyzed.

### Example 5

### Intracerebral Injection of Tamoxifen to PLP/CreER^{T}

### ROSA26-eGFP-DTA Mice

To induce localized demyelination in the PLP/CreER^{T}; ROSA26-eGFP-DTA mouse, tamoxifen is injected into the mouse by stereotaxic injection. A stereotaxic instrument is used to implant an injection probe for delivering the tamoxifen solution through a cannula to the preselected location, which is defined by co-ordinates from the mouse brain atlas. Approximately 10 to 100 microgram/day, for 1 to 3 days, is administered to the mouse continuously.

The intracerebral injection is performed in younger mice (5-6 weeks of age) and older mice (6-9 months of age). The mice are characterized by CC1 immunostaining, CD11b staining, EM, toluidine blue staining, Mbp and Plp gene expression, rotarod assay, analysis of the gait by the treadmill DigiGait assay, and SEP assay. Immunohistochemistry is also performed. The effect and any differences, such as time for recovery or extent of recovery, of the younger versus older mice are analyzed.

### Example 6

### Repeated Demyelination and Remyelination of PLP/CreER^{T}

### ROSA26-eGFP-DTA Mice

To determine the remyelination potential and phenotype reversal potential of the mice, tamoxifen treated PLP/CreER^{T}; ROSA26-eGFP-DTA younger mice (5-6 weeks of age) and older mice (6-9 months of age) that have recovered (such as described in Example 3) or after an amount or dose of tamoxifen that was administered as determined in Example 4, by IP. In one group of mice, the second injection of tamoxifen is 120 days PI.

The mice are characterized by CC1 immunostaining, CD11b staining, EM, toluidine blue staining, Mbp and Plp gene expression, rotarod assay, analysis of the gait by the treadmill DigiGait assay, and SEP assay. Immunohistochemistry is also performed. The results are also compared to mice that were treated with tamoxifen only once, to determine if the recovery time is longer, the recovery is not as robust, or the demyelination and phenotypes are more severe in comparison to the mice that have been treated once. The effect and any differences, such as time for recovery or extent of recovery, of the younger versus older mice are also analyzed.

The same experiment is repeated, where tamoxifen is administered focally (see Example 5) as is LPS.

### Example 7

### Administration of LPS and Tamoxifen to PLP/CreER^{T}

### ROSA26-eGFP-DTA Mice

The PLP/CreER^{T}; ROSA26-eGFP-DTA younger mice (5-6 weeks of age) and older mice (6-9 months of age) are administered tamoxifen, an amount or dose as described in Example 1 or determined in Example 4, by IP. A single dose of LPS of 200 micrograms is administered IP at 7 and 14 dpi, or by injecting a daily dose of 10 micrograms for 7 continuous days starting at 7 or 14 dpi, prior to the animal exhibiting phenotypic characteristics of a demyelination disorder.

The mice are characterized by CC1 immunostaining, CD11b staining, EM, toluidine blue staining, Mbp and Plp gene expression, rotarod assay, analysis of the gait by the treadmill DigiGait assay, and SEP assay. Immunohistochemistry is also performed. The influx of immune cells into the brain is also determined by looking at the following markers: granulocyte/neutrophil antigen 4/7, T cell antigen CD3, and IBA1. The effect and any differences, such as time for recovery or extent of recovery, of the mice that were treated with LPS are compared to mice that were administered tamoxifen but not treated with LPS. The mice treated with LPS can have a faster recovery time. The effect and any differences, such as time for recovery or extent of recovery, of the younger versus older mice are also analyzed.

The same experiment is repeated, where tamoxifen is administered focally (see Example 5) as is LPS. The same experiment is also performed, where there are multiple administrations of tamoxifen (see Example 6), where LPS is also administered for each administration of tamoxifen.

### Example 8

### Integrity of Blood Brain Barrier in PLP/CreER^{T}

### ROSA26-eGFP-DTA Mice

The PLP/CreER^{T}; ROSA26-eGFP-DTA younger mice (5-6 weeks of age) and older mice (6-9 months of age) are administered tamoxifen, an amount or dose as described in Example 1 or determined in Example 4, by IP. To test the integrity of the blood brain barrier (BBB), Evan blue dye is injected IP (2%, 4 ml/kg of body weight) in the mice at 48 hours prior to sacrifice. The distribution of the blue dye is analyzed in the brain parenchyma to determine the integrity of the BBB.

### Example 9

### Screening of a Biologically Active Agent

The PLP/CreER^{T}; ROSA26-eGFP-DTA mice is administered tamoxifen as described in Example 1 or an amount or dosage as determined in Example 4, by IP. A candidate agent is administered by IP to the mouse 14 days PI, prior to the animal exhibiting phenotypic characteristics of a demyelination disorder.

The mice are characterized by rotarod assay and analysis of the gait by the treadmill DigiGait assay at various time points. The agent is selected for further development as a therapeutic when the mouse recovers its motor control faster than the PLP/CreER^{T}; ROSA26-eGFP-DTA mice that are administered tamoxifen but not the candidate agent.

The present invention is not limited to the embodiments described above, but is capable of modification within the scope of the appended claims. Those skilled in the art will recognize, or be able to ascertain using no more than routine experimentation, many equivalents of the specific embodiments of the invention described herein. Numerous variations, changes, and substitutions will now occur to those skilled in the art without departing from the invention. It should be understood that various alternatives to the embodiments of the invention described herein can be employed in practicing the invention. It is intended that the following claims define the scope of the invention and that methods and structures within the scope of these claims and their equivalents be covered thereby.

1. A non-human transgenic animal, said animal comprising:
a) a first heterologous nucleotide sequence operably linked to a glial-cell specific promoter,
wherein said first heterologous nucleotide sequence encodes a first heterologous protein and said first heterologous nucleotide sequence is stably expressed in said animal;
b) a second heterologous nucleotide sequence encoding a second heterologous protein,
wherein said second heterologous protein is expressed upon activation of said first heterologous protein and induces death of a non-proliferating glial cell,
wherein a initial activation of said first heterologous protein induces death of said non-proliferating glial cells in said animal and results in demyelination in said animal and yields one or more phenotypic changes characteristic of a demyelination condition; and
wherein said one or more phenotypic changes is reversed after initial activation of said first heterologous protein.

2. The non-human transgenic animal of claim 1, wherein said animal is a mammal.

3. The non-human transgenic animal of claim 1, wherein said animal is selected from the group consisting of a mouse, rat, guinea pig, rabbit, dog, cat, pig, and monkey.

4. (canceled)

5. The non-human transgenic animal of claim 4 wherein said mouse is at least 5 weeks old, 4 to 6 months old or is an adult.

6. (canceled)

7. (canceled)

8. The non-human transgenic animal of claim 1, wherein said non-proliferating glial cell is in the central nervous system (CNS).

9. The non-human transgenic animal of claim 1, wherein said non-proliferating glial cell is in the peripheral nervous system (PNS).

10. The non-human transgenic animal of claim 1, wherein activation of said first heterologous protein is inducible.

11. The non-human transgenic animal of claim 1, wherein said activation is induced by an exogenous agent.

12. The non-human transgenic animal of claim 1, wherein said activation is recombination.

13. The non-human transgenic animal of claim 1, wherein said first heterologous protein is a recombinase.

14. The non-human transgenic animal of claim 13, wherein said recombinase is a Cre recombinase or variant thereof.

15. The non-human transgenic animal of claim 14, wherein said variant is a fusion protein.

16. The non-human transgenic animal of claim 15, wherein said fusion protein is of Cre recombinase and a mutated ligand binding domain of an estrogen receptor.

17. The non-human transgenic animal of claim 11, wherein said exogenous agent is tamoxifen or an analog thereof.

18. The non-human transgenic animal of claim 15, wherein said fusion protein is CreER^{T} or CreER^{T2}.

19. The non-human transgenic animal of claim 1, wherein said glial-cell specific promoter is a promoter of a gene selected from the group consisting of proteolipid protein (PLP), myelin basic protein (MBP), oligodendrocyte specific protein (OSP), myelin oligodendrocyte glycoprotein (MOG), ceramide galactosyltransferase (CGT), myelin associated glycoprotein (MAG), oligodendrocyte-myelin glycoprotein (OMG), cyclic nucleotide phosphodiesterase (CNP), NOGO, myelin protein zero (MPZ), peripheral myelin protein 22 (PMP22), or protein 2 (P2).

20. The non-human transgenic animal of claim 1, wherein said glial-cell specific promoter is a promoter of a gene selected from the group consisting of PLP, MBP, and CNP.

21. The non-human transgenic animal of claim 1, wherein said second heterologous protein induces cell death.

22. The non-human transgenic animal of claim 1, wherein said second heterologous protein is an exotoxin.

23. The non-human transgenic animal of claim 22, wherein said exotoxin is a diptheria toxin or subunit thereof.

24. The non-human transgenic animal of claim 23, wherein said diptheria toxin subunit is the A subunit.

25. The non-human transgenic animal of claim 1, wherein said non-proliferating glial cell is an oligodendrocyte or Schwann cell.

26. The non-human transgenic animal of claim 1, wherein said one or more phenotypic changes characteristic of a demyelination condition is selected from the group consisting of wobbly gait, hind limb paralysis, tremors, weight loss, ataxia, decreased motor control, balance, CNS conduction, and any combination thereof.

27. (canceled)

28. (canceled)

29. (canceled)

30. The non-human transgenic animal of claim 1, wherein said one or more phenotypic change is reversed about 35 days or more after said activation of said first heterologous protein.

31. The non-human transgenic animal of claim 1, wherein said one or more phenotypic change is reversed about 70 days or more after said activation of said first heterologous protein.

32. A cell of said non-human transgenic animal of claim 1, wherein said cell comprises:
a) a first heterologous nucleotide sequence operably linked to a glial-cell specific promoter,
wherein said first heterologous nucleotide sequence encodes a first heterologous protein and said first heterologous nucleic acid is stably expressed in said cell; and
b) a second heterologous nucleotide sequence encoding a second heterologous protein,
wherein said second heterologous protein is expressed upon activation of said first heterologous protein and induces death of said cell upon activation of said first heterologous protein.

33. (canceled)

34. (canceled)

35. (canceled)

36. A method of selecting an agent that promotes reversal of one or more phenotypic changes characteristic of a demyelination condition comprising:
a) activating said first heterologous protein in said non-human transgenic animal of claim 1;
b) administering a candidate agent to said animal;
c) determining one or more phenotypic changes characteristic of a demyelination condition in said animal; and
d) selecting said agent when said one or more phenotypic changes is reversed more quickly as compared to a control animal not administered said candidate agent.

37. (canceled)

38. (canceled)

39. (canceled)

40. (canceled)

41. (canceled)

42. (canceled)

43. A method of selecting an agent that promotes remyelination comprising:
a) activating said first heterologous protein in said non-human transgenic animal of claim 1;
b) administering a candidate agent to said animal;
c) determining remyelination in said animal; and
d) selecting said agent when said animal displays increased remyelination as compared to a control non-transgenic animal not administered said candidate agent.

44-70. (canceled)
